(19) United States
(12) Patent Application Publication
(10) Pub. No.: US 2024/0029714 A1
(43) Pub. Date: Jan. 25, 2024

(54) SPEECH SIGNAL PROCESSING AND SUMMARIZATION USING ARTIFICIAL INTELLIGENCE

(71) Applicants: Bharath Chintagunta, Palo Alto, CA (US); Namit Katariya, Palo Alto, CA (US); Xavier Amatriain, Los Gatos, CA (US); Anitha Kannan, Saratoga, CA (US); Varun Nair, San Francisco, CA (US); Elliot Schumacher, Pandora, OH (US); Mika Wang, Palo Alto, CA (US); Ilya Valmianski, Palo Alto, CA (US); Maksim Ereemev, Palo Alto, CA (US)

(72) Inventors: Bharath Chintagunta, Palo Alto, CA (US); Namit Katariya, Palo Alto, CA (US); Xavier Amatriain, Los Gatos, CA (US); Anitha Kannan, Saratoga, CA (US); Varun Nair, San Francisco, CA (US); Elliot Schumacher, Pandora, OH (US); Mika Wang, Palo Alto, CA (US); Ilya Valmianski, Palo Alto, CA (US); Maksim Ereemev, Palo Alto, CA (US)

(21) Appl. No.: 18/351,431

(22) Filed: Jul. 12, 2023

Related U.S. Application Data
(60) Provisional application No. 63/396,503, filed on Aug. 9, 2022.
(60) Provisional application No. 63/396,509, filed on Aug. 9, 2022.
(60) Provisional application No. 63/388,566, filed on Jul. 12, 2022.
(60) Provisional application No. 63/522,112, filed on Jun. 20, 2023.

(51) Int. Cl. G10L 15/06; G10L 15/16; G10L 15/183

(52) U.S. Cl. CPC G10L 15/063; G10L 15/16; G10L 15/183; G10L 2015/0631; G10L 15/22

(57) ABSTRACT

An apparatus for speech signal processing using artificial intelligence comprises: a microphone configured to receive speech and convert the received speech to a digital speech signal; at least one processor; and a non-transitory computer-readable medium having stored thereon instructions to cause the least one processor to execute the method of speech signal processing using artificial intelligence. The method comprises: receiving the digital speech signal; converting the speech signal to text; labelling, with at least one machine learning model, components of the text; and generating, with the at least one machine learning model, with the labelled components, at least one of a care plan or summary.

### CROSS REFERENCE TO RELATED APPLICATIONS

This application claims priority to an incorporates by reference U.S. Provisional Patent Application Nos. 63/396,503 filed Aug. 9, 2022; 63/396,509 filed Aug. 9, 2022; 63/388,566 filed Jul. 12, 2022; and 63/522,112 filed Jun. 20, 2023.

### TECHNICAL FIELD

This disclosure relates to data processing and more specifically, but not exclusively, speech signal processing using artificial intelligence.

### BACKGROUND

Data processing can include processing of speech signal processing, linguistics, language translation, and audio compression/decompression. Further, this data processing can be performed by artificial intelligence. However, often lack of available annotations hampers the ability to learn effective artificial intelligence classification models.

### SUMMARY

An apparatus for speech signal processing using artificial intelligence comprises: a microphone configured to receive speech and convert the received speech to a digital speech signal; at least one processor; and a non-transitory computer-readable medium having stored thereon instructions to cause the least one processor to execute the method of speech signal processing using artificial intelligence. The method comprises: receiving the digital speech signal; converting the speech signal to text; labelling, with at least one machine learning model, components of the text; and generating, with the at least one machine learning model, with the labelled components, at least one of a care plan or summary.

### BRIEF DESCRIPTION OF THE DRAWINGS

FIG. 1 illustrates a system according to an example embodiment.

FIG. 2 illustrates a de-identified patient-medical professional dialog coded with the predictions from an example embodiment. Note that different multiple labels may be present in the same turn of the conversation.

FIG. 3 illustrates an example method of bootstrapping.

FIG. 4 illustrates an example of clustering of Sentences Predicted as “Summarization” after a First Round of Training.

FIG. 5 illustrates improvement in label purity with each round iteration the example bootstrapping method.

FIG. 6 illustrates cosine similarity of same- and different-class pairs for each class.

FIG. 7(a) and FIG. 7(b) illustrate Example conversation segments corresponding to care plan and corresponding instructions. Arrows represent semantic relationship between the dialogue sentence and instruction. Note that these relationships between the dialog and the instructions are not available in a dataset.

FIG. 8(a) and FIG. 8(b) illustrate empirical concept marginal probabilities and utilization rates estimated from the dataset.

FIG. 9(a) and FIG. 9(b) illustrate relative errors in the utilization rates for different semantic types plotted as a function of the frequency of the semantic type. The trend-line and uncertainty are computed with a linearly interpolated moving average window.

FIGS. 10(a), (b) and (c) illustrate entropy of the conditional distribution p(y|y<t, x) with respect to different α values. Filled regions denote the standard deviation across training runs.

FIG. 11 illustrates a routine for training models on a mix of GPT-3-ENS synthesized and human labeled data.

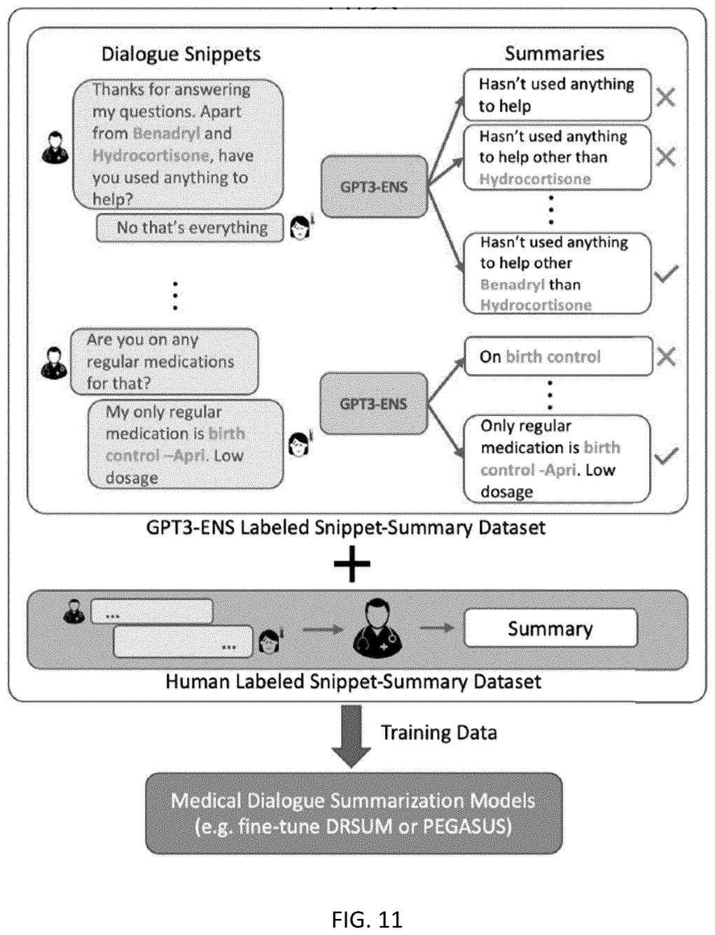

FIG. 12 illustrates doctor evaluation of which among GPT-3 and GPT-3-ENS summaries they considered “best” showing that an example embodiment is a better approach for labeling.

FIG. 13(a) and FIG. 13(b) illustrate Doctor evaluation of amount of medical information covered by summaries provided by PEGASUS models and which ones they considered “best”.

FIG. 14(a) and FIG. 14(b) illustrate doctor evaluation of amount of medical information covered by summaries provided by DRSUM models and which ones they considered “best”.

FIG. 15 illustrates an example embodiment that utilizes a multi-stage approach for medical dialogue summarization with GPT-3 that improves upon naive summarization. The approach utilizes intermediate model calls to extract medical concepts that inform summarization generation.

FIG. 16 illustrates results of human expert evaluations show the example embodiment of FIG. 15 (5-shot) is preferred 66% to 34% over a single-prompt, 0-shot naive summarization baseline.

FIG. 17 illustrates an example routine for speech processing with an artificial intelligence.

FIG. 18 illustrates an example routine for speech processing with an artificial intelligence.

FIG. 19 illustrates an example routine for speech processing with an artificial intelligence.

FIG. 20 illustrates an example routine for speech processing with an artificial intelligence.

FIG. 21 illustrates an example routine for speech processing with an artificial intelligence.

FIG. 22 is a block diagram illustrating a software architecture, which can be installed on any one or more of the devices described herein.

FIG. 23 is a diagrammatic representation of the machine within which instructions (e.g., software, a program, an application, an applet, an app, or other executable code) for causing the machine to perform any one or more of the methodologies discussed herein may be executed.

### DETAILED DESCRIPTION

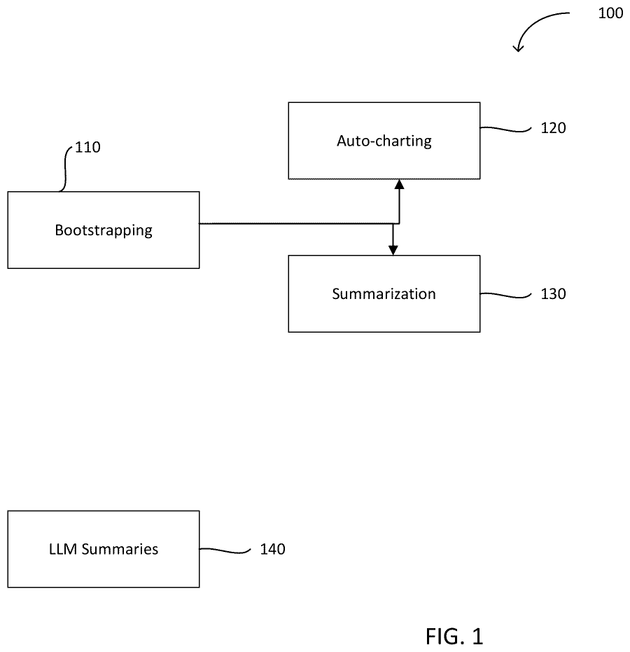

FIG. 1 illustrates a system 100 according to an example embodiment. The system 100 comprises a bootstrapping component 110, an auto-charting component 120, a summarization component 130 and a large language model (LLM) summaries component 140.

The bootstrapping component 110 uses a two-step approach for bootstrapping labels with minimal human annotation. Labels can include, for example, in a medical conversation, “history taking”, “summarization”, “education”, “care plan” or “other.” First, the component 110 use heuristics to generate turn-level pseudolabels and train a transformer-based model, which is then applied on sentences to create noisy sentence-level labels. Second, the component 110 iteratively refines sentence-level labels using a cluster-based human-in-the-loop approach. Each iteration requires only a few dozen annotator decisions. After labeling by the component 110, auto-charting of after-visit care instructions can be generated with the auto-charting component 120. The component 120 operates on rare tokens that appear in both the source and reference sequences, and which, when missed during generation, can hamper the factual correctness of the generated text. The component 120 (a) identifies what rare tokens that appear in both source and reference are important and (b) uplift their conditional probability. The component 120 uses a “utilization rate” that encodes knowledge and serves as a regularizer by maximizing the marginal probability of selected tokens. The component 120 then can generate after-visit care instructions based on patient-doctor dialogues.

Alternatively, or in addition, the summarization component 130 can then summarize a dialogue, e.g., a medical dialogue. The component 130 uses an algorithm to create synthetic training data with an focus on capturing medically relevant information. The component 130 can use a generative machine learning model, e.g., GPT-3, as the backbone of the algorithm and scale 210 human labeled examples to yield results comparable to using 6400 human labeled examples (˜30×) leveraging low-shot learning and an ensemble method. The component 140 summarizes medical conversation summarization by discretizing the task into several smaller dialogue understanding tasks that are sequentially built upon. The component 140 identifies medical entities and their affirmations within the conversation to serve as building blocks. The component then dynamically constructs few-shot prompts for tasks by conditioning on relevant patient information and use a generative machine learning model (e.g., GPT-3) as the backbone.

### Bootstrapping Component 110

Recent growth in telemedicine has led to a dramatic expansion in text-based chat communications between patients and medical professionals. This creates new opportunities for improving medical professional workflows through the introduction of natural language understanding (NLU) systems for providing real-time decision support and automating electronic health record (EHR) charting. EHR charting automation is especially important, as charting is a significant source of medical professional burnout and the charted information can oftentimes be directly extracted from medical professional-patient dialogue. For example, the History of Present Illness (HPI) section of the progress note can be derived from the history-taking discussion in the dialogue, while the Care Plan section can be derived from the care plan discussion. Auto-charting tasks benefit significantly from proper contextualization of the dialogue and in particular its discourse structure.

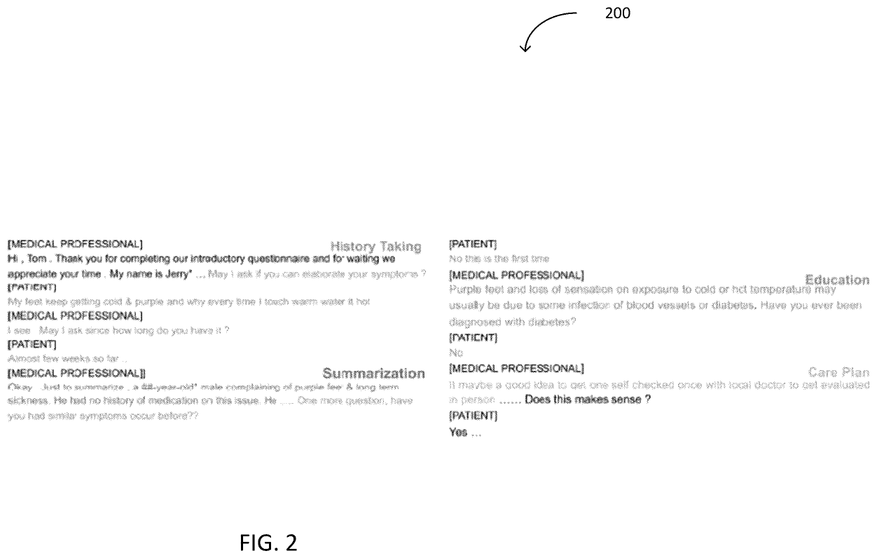

A conversational dialogue between a medical professional and a patient has fundamental medical-related discourses. The discourse units include (a) gathering the patient's history of present illness (labeled “History taking”), (b) confirming captured patient symptomatology (labeled “Summarization”), (c) educating the patient (labeled “Education”) and (d) communicating a treatment plan (labeled “Care plan”). There are also other non-medical discourses, such as the expression of empathy and discussion of technical difficulties. Dialog does not explicitly codify this structure. FIG. 2 shows an abridged dialogue 200 in the sentences are labeled as predicted by the model introduced. Note that both the patient and the medical professional may use language with high lexical overlap during each part of the dialogue 200. For example, a medication discussion can be part of history taking (e.g. “Were you taking Amoxicillin when you developed abdominal pain?”), summarization (e.g. “You developed abdominal pain after taking Amoxicillin”), education (e.g. “You developed abdominal pain due to taking Amoxicillin”), and care plan (e.g. “To help with abdominal pain stop taking Amoxicillin”). In addition, within a single turn of the dialog, multiple sections can co-occur, e.g. a medical professional may educate the patient while gathering history by saying, “Acidic foods increase acid reflux. Are you eating acidic foods such as citrus fruits?”.

We formulate the problem of inferring conversation discourse structure as the problem of fine-grained discourse label assignment: Given a medical professional-patient dialogue, how can we assign every sentence in every turn of the dialogue to the correct semantic labe?

A direct approach to this problem would be to treat this as a classification task, assuming access to a large collection of labeled training data. There are two main challenges with getting access to a large labeled training set. First, a typical dialogue contains tens to hundreds of sentences and would be too distracting for a medical professional to annotate during the encounter. Offline human annotation is also expensive because the annotators need sufficient medical knowledge. The data needs to obey privacy rules around patient health information (PHI). In addition, the discourse classes are highly imbalanced, e.g. there is far more history taking than any other class, implying that we need to label a large number of encounters to achieve sufficient representation of minority classes.

To overcome these challenges, an example embodiment trains a highly accurate machine learning (ML) model with minimal amounts of human-generated labels through iterative label-bootstrapping. The example embodiment leverages two insights. First, sentences in a turn tend to share labels, and while it is hard to come up with heuristics to label individual sentences, it is much easier to come up with heuristics that label an entire turn. The example embodiment uses this insight to build a noisy turn-level labels data set and train a language model to classify turn-level labels. The example embodiment then applies the turn level model to label individual sentences within the turn, creating noisy sentence-level labels on which we train a sentence level model. Second, even if the model is poor, the latent space representations it produces are still highly relevant to the labeling task. According, an example embodiment employs an iterative human-in-the-loop cluster-based pseudolabeling strategy, starting with labeled data generated by the turn-level model. The clustering strategy introduces variability in samples across iterations by enabling intermixing high confidence predictions with low-confidence ones and choosing only class-specific ‘pure’ clusters through a simple human-in-the-loop evaluation.

Evaluation of the results on an expert-annotated dataset of 100 dialogues and find that although the initial pseudolabels have a low accuracy of 69.5%, while the iterative refinement approach can boost accuracy to 82.5%. Further, the latent space representations of each class become both more tightly clustered and more separable between different classes, which may imply higher generalizability.

We are interested in classifying dialogue turns and also each sentence within a turn into higher-level medical categories (history taking, summary, education, care plan) that can loosely serve as intents. Also, within a single dialog turn, these categories interleave (e.g. history taking and education), making the problem of segmentation challenging. An example embodiment bootstraps a small amount of training data using a coarse-grained turn-based classification model and then introduces a pseudolabeling strategy that leverages clustering and a human-in-the-loop to improve model performance iteratively.

### Problem Setup

A dataset of medical dialogues between medical professionals and patients using our virtual primary care service may be used. A highly abridged example of such dialogue 200 can be seen in FIG. 2. Each dialogue, D∈D, is an ordered set of dialogue turns Tᵢ∈D, which are themselves ordered sets of individual sentences Sᵢⱼ∈Tᵢ. In addition, for each turn there is a user identity Uᵢ∈ (“patient”, “medical professional”). An example embodiment will classify each medical professional generated sentence (Sᵢⱼ: Uᵢ=“medical professional”) into one of the following five sections (Lᵢⱼ∈ [symbol] ):
1. History taking: Sentences where the medical professional is asking the patient about their current illness including symptoms, prior medical history, and medications they may be taking.
2. Summarization: Sentences where the medical professional provides the patient the (medical professional's) understanding of relevant patient symptomatology.
3. Education: Sentences where the medical professional educates the patient about the medical issues facing the patient.
4. Care plan: Sentences where the medical professional suggests a course of action for the patient; this may include actions the patient should take or medical professional orders such as medication prescriptions and lab workups.
5. Other: Sentences that do not belong to the aforementioned classes.

Starting with unlabeled data of dialogues, a classifier is trained that can accurately label each sentence in every dialogue into one of the five classes above. We assume minimal availability of just-in-time oracle for labeling (e.g. annotator).

The example embodiment may train an effective sentence-level classification model Mₛₑₙₜ: SᵢⱼD→Lᵢⱼ, Lᵢⱼ∈ [symbol] , Sᵢⱼ∈Tᵢ∈D, which maps the sentence Sᵢⱼ in the context of dialogue D to a label Lᵢⱼ. In order to learn this supervised model, we introduce a pseudolabeling strategy that produces labeled data needed for training Mₛₑₙₜ. Instead of using humans to annotate specific sentences Sᵢⱼ, we exploit either textual structure (sentences in a given turn tend to share labels) or latent space structure (sentences that are close together in a relevant latent space tend to share labels) to label many sentences at once.

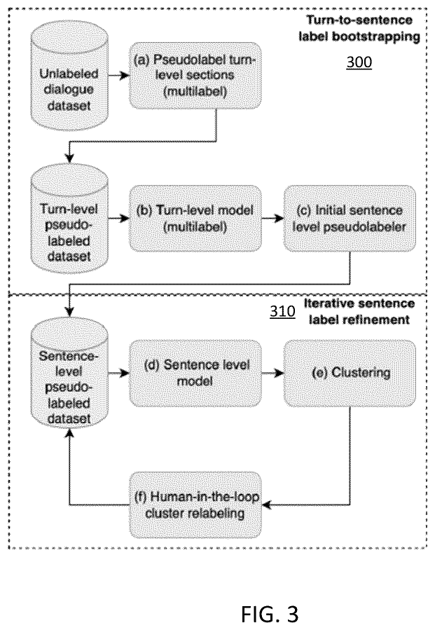

This pseudolabeling operates in two steps as shown in FIG. 3:
1. Turn-to-sentence label bootstrapping 300: Use task-specific heuristics to create turn-level pseudolabels and train a turn-level model, which is then applied to create sentence-level pseudolabels. Bootstrapping 300 comprises create (a) turn-level pseudolabeler using a mix of clustering, heuristics, and human labeling, (b) use this to train a turn-level model, and (c) use the turn-level model to help pseudolabel individual sentences.
2. Iterative sentence label refinement 310: Train a sentence-level model, cluster the sentence-level model representations conditioned on the predicted label, and then use a oracle to relabel each cluster based on its purity. Refinement 310 comprises (d) train a sentence-level model and (e) iteratively refine sentence-level pseudolabels by clustering the sentence-level model representations and (f) relabeling clusters using human-in-the-loop.

### Turn-to-Sentence Label Bootstrapping 300

It is difficult to develop good heuristics for labeling individual sentences in a dialogue, as many sentences are incomplete or heavily rely on the surrounding context. However, sentences in a single turn tend to share labels, and often at least one sentence will be amenable to heuristics. To exploit this an example embodiment uses heuristics for creating turn-level multilabel annotations Lᵢ^{turn}=Uⱼ Lᵢⱼ. The labels created by these heuristics to train a turn-level multilabel model Mₜᵤᵣₙ: Tᵢ, D→Lᵢ^{turn} is then used to generate sentence-level labels by being applied directly on sentences instead of entire turns (Lᵢⱼ⁰←Mₜᵤᵣₙ(Sᵢⱼ, D), the 0 superscript refers to this being the initial set of sentence-level pseudolabels).

### Iterative Sentence Label Refinement 310

An iterative algorithm, algorithm 1, for refinement of sentence-level pseudolabels is shown below. At each iteration, based on previous iteration's labels, Lᵢⱼ^{k}, a new model is trained Mₛₑₙₜ^{k}:SᵢⱼD→\hat{L}ᵢⱼ^{k}, Eᵢⱼ^{k}, where Eᵢⱼ^{k} is a fixed size embedding of (Sᵢⱼ, D), e.g. mean pooling of the penultimate layer of Mₛₑₙₜ^{k}. The embeddings are then clustered using CL:{(\hat{L}ᵢⱼ^{k}, Eᵢⱼ^{k})}→{Cₙ}.

Input: Dialogue dataset D_{dev}
Current iteration model Mₛₑₙₜ^{k}
Current iteration pseudolabels Lᵢⱼ^{k}
Clustering algorithm CL
Cluster label oracle H

Output: Next iteration pseudolabels, {Lᵢⱼ^{k+1}}
1 ∀D∈ [symbol] _{dev}, Sᵢⱼ∈D, (\hat{L}ᵢⱼ^{k}, Eᵢⱼ^{k})←Mₛₑₙₜ^{k}(Sᵢⱼ, D)
2 {Cₙ}←CL({(\hat{L}ᵢⱼ^{k}, Eᵢⱼ^{k})})
3 Lᵢⱼ^{k+1}←H(Cₙ) arg whereₙ Eᵢⱼ^{k}∈Cₙ
4 return {Lᵢⱼ^{k+1}: Lᵢⱼ^{k+1}≠Mixed}
Algorithm 1: Pseudocode for iterative cluster refinement of sentence level models

Access to an oracle can provide the label to be assigned to all elements of that cluster, including a label of None that removes examples in that cluster from being used in training the next round's model (Mₛₑₙₜ^{k}). While we can compute cluster label purity quantitatively, this requires access to a large amount of labeled data, a problem that an example embodiment may address. Therefore, we use a human annotator as an oracle that assigns the label to the cluster for practicality. The labeling is efficient and labor-friendly because, in each cluster containing hundreds of thousands of examples, we only need to label a few that are typically in the extremes of a cluster when visualized graphically. From experiments, we found the best clustering strategy is to project Eᵢⱼ to a lower-dimensional space through the use of PCA and UMAP and then cluster the embeddings separately for each the predicted label. This significantly improves the human-judged purity of the clusters.

### Experimental Details

### Dataset

We use a dataset with 60,000 medical professional-patient encounters containing over 900,000 dialogue turns and 3,000,000 sentences collected on a virtual primary care platform. We do not have any labels for these encounters.

To construct a test set, we randomly sampled 100 encounters (not used for training or validation) for which we procured human labels for all medical professional written sentences (3,102 sentences). We use the [20%,80%] interval of the cumulative distribution of the total number of medical professional written sentences to sample these encounters. In the human-labeled dataset, the distribution of sections on the sentence and turn level, are respectively: summarization: 3.6%, 2.6%; history taking: 26.5%, 31.7%; education: 5.3%, 8.4%; care plan: 4.1%, 7.9%; other: 60.3%, 49.3%.

### Turn-to-Sentence Label Bootstrapping

We start by generating turn-level labels with heuristics, unsupervised clustering, and human annotations for mixed classes obtained after clustering. We then train a turn-level multilabel classification model on these pseudolabels and apply this model at the sentence level.

Turn-level pseduolabeling We build a labeled dataset at the turn level by clustering the turns and manually assigning labels to the clusters.

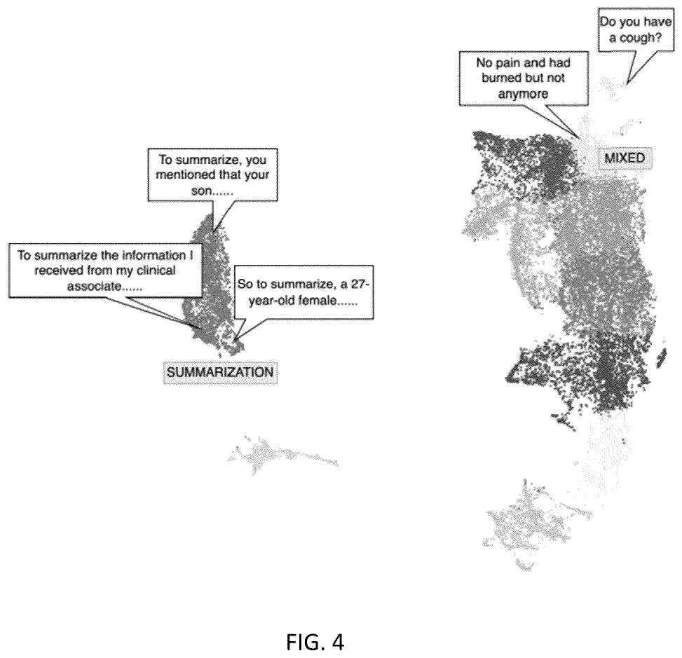

For clustering, we first embed the turns into fixed-sized representations by mean-pooling the final layer of the off-the-shelf DeCLUTR sentence encoder. We project the 768D original embedding space to 250D via PCA and then project via UMAP to 50D. We then cluster these 50D representations using the k-means++algorithm and determine the number of clusters using the elbow method) (in our dataset, this number was 10). We manually label the resulting clusters by examining ten distant data points from the cluster as shown in FIG. 4. We label the clusters into precisely one of the discourse classes or Other or mixed.

We procured human labels for 5000 turns from a mixed cluster that was predominantly education and care plan labeled as “Education”, “Care plan” and “Other.”, since the turn model did not produce any dominant clusters specifically for education and care plan. We use a rule-based labeler for identifying “Summarization” turns by string matching one of the following strings [‘summar’, ‘sum up’] that medical professionals use.

Turn-level model to generate sentence psuedolabel. We construct the dataset for the turn-level model by assigning the same label as the cluster after removing all mixed clusters. We then train Mam, a multi-label classifier on top of DeCLUTR using this turn-level labeled set. The classification head consists of a single feed-forward layer with sigmoidal activation for each label.

To create the initial sentence level labels, we apply the turn-level model on each sentence and assign labels according to Algorithm 1 in the Appendix.

### Iterative Sentence Label Refinement

Sentence-level model. The input to this model is the dialogue turn that contains the target sentence. We mark the target sentence with tokens (START) and (END). The model itself comprises a transformer language model DeCLUTR sentence encoder, with a classification head comprising a single feed-forward layer with a softmax activation.

Clustering sentence-level model To cluster sentence-level embeddings, we use a similar approach to the one described in turn-level clustering, except we apply the kmeans++algorithm independently for each predicted label.

FIG. 4 shows the visualization of clusters predicted to be part of “Summarization.” Each cluster is manually assigned its label (often simply staying with the original predicted label) by examining several distant data points (sentences).

Details of relabeling between rounds. Table 1 shows the number of clusters relabeled and the new label assigned. We can see that most relabeling was moving clusters to “Mixed” label, in which case examples in that cluster were not used for the subsequent round of model training (however, they would still be used for subsequent clustering and relabeling).

TABLE 1
Cluster relabeling at between each round. Each row represents
the original label of the clusters. In each cell, the ratio
to the left of the arrow represents the number of clusters
(out of total for that label) that were relabeled. The text
on the right represents the new assigned label. Clusters assigned
“Mixed” are not used in the training of the model in
the next round (but are still used in subsequent rounds).

| | Round 1→2 | Round 2→3 | Round 3→4 |
|---|---|---|---|
| History taking | — | 1/10→Mixed | 3/10→Mixed |
| Summarization | 2/10→Mixed | 1/10→Mixed | 3/10→Mixed |
| | | 1/10→Other | |
| Education | 1/10#Mixed | 6/10#Mixed | 3/10#Mixed |
| | | | 1/10#Other |
| Care plan | 1/10#Mixed | 3/10#Mixed | 6/10#Mixed |
| Other | 7/15#Mixed | 3/10#Mixed | 6/10#Mixed |

### Implementation Details

All models discussed are trained in Pytorch 1.10.2+cu102 with the language models implemented using HuggingFace Transformers library. The weights for the DeCLUTR models were using the JOHNGIORGI/DECLUTR-BASE checkpoint. For training, we used the Adam optimizer with learning rate 2e⁻⁵ 475 and a scheduler with warm-up steps of total training steps/5. We set batch size as 12. PCA and kmeans were implemented using scikit-learn 0.24.2 package, while UMAP used the umap-learn 0.5.1 package.

### Results

### Main Results: Sentence-Level Model Performance

Table 2 provides main results, comparing F1 and accuracy scores from each training round of the sentence-level model. The overall performance increased from an accuracy of 69.5% to 82.5%. This three-fold improvement of the F1 scores shows that the example iterative approach can improve labeling quality (and hence the model) even when the initial labels are quite noisy. The “Summarization” class has the most improvement (F1 score from 0.18 to 0.65). The sentences in this class are hard to identify solely from the turn-level-model-based pseudo-labeling because they vary in structure. The pseudolabeler successfully labels the sentences that contain “to summarize.” The iterative clustering-based labeling introduces less-confident predictions that are semantically similar to more confident ones to improve the overall identifiability.

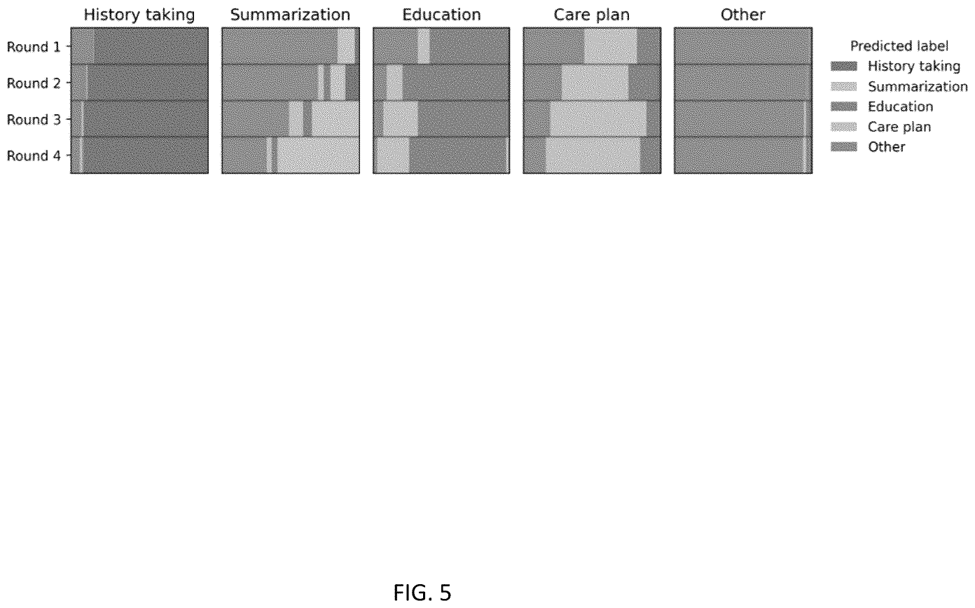

To understand the rationale for these metrics, FIG. 5 provides a graphical representation of the errors the model makes. Each column represents the human-assigned true label in this figure, and each row represents the proportion of the predicted labels in each true label for each training round. The two sections that see the significant F1 score uplift, “History taking” and “Summarization,” start with a significant confusion with the “Other” section, which gradually decreases. Even though the additional iterations did not improve the care plan and education classes, their overall confusion changed between rounds. Initially, both “Education” and “Care plan” were confused with the “Other” section, while in later rounds, they were confused with each other. We expect this inter-class confusion as they can be hard to differentiate even for human annotators, e.g. “It is recommended that a person having a fever should drink more water.” could be annotated as either “Education” or “Care plan,” depending on the context.

TABLE 2
Sentence-level model performance: F1 scores and
accuracy after each round of iterative training
F1 score

| Class | Round 1 | Round 2 | Round 3 | Round 4 |
|---|---|---|---|---|
| Summarization | 0.18 ± 0.00 | 0.19 ± 0.11 | 0.47 ± 0.06 | 0.65 ± 0.02 |
| History Taking | 0.89 ± 0.01 | 0.9 ± 0.00 | 0.92 ± 0.00 | 0.93 ± 0.01 |
| Education | 0.70 ± 0.01 | 0.69 ± 0.02 | 0.69 ± 0.02 | 0.65 ± 0.02 |
| Care Plan | 0.55 ± 0.02 | 0.56 ± 0.03 | 0.57 ± 0.01 | 0.55 ± 0.02 |
| Other | 0.90 ± 0.00 | 0.92 ± 0.00 | 0.93 ± 0.00 | 0.93 ± 0.00 |
| Multi-class Accuracy | 69.5% ± 0.00 | 74.1% ± 0.00 | 80.4% ± 0.01 | 82.5% ± 0.01 |

* Accuracy is on the four functional classes only

TABLE 3
Turn-based inference improved with sentence-level model. The column ‘turn-
level’ provides the F1-score of the model from which sentence level model
was bootstrapped. The remaining columns Round 1-4 show the F1-score when
we pool sentence-level predictions in a turn using sentence-level model.

| | F1 score | | | | |
|---|---|---|---|---|---|
| Class | Turn-level | Round 1 | Round 2 | Round 3 | Round 4 |
| Summarization | 0.22 ± 0.00 | 0.22 ± 0.00 | 0.25 ± 0.03 | 0.69 ± 0.04 | 0.66 ± 0.04 |
| History Taking | 0.37 ± 0.00 | 0.84 ± 0.02 | 0.83 ± 0.01 | 0.86 ± 0.01 | 0.87 ± 0.01 |
| Education | 0.61 ± 0.01 | 0.77 ± 0.02 | 0.69 ± 0.05 | 0.73 ± 0.02 | 0.65 ± 0.04 |
| Care Plan | 0.31 ± 0.04 | 0.55 ± 0.02 | 0.55 ± 0.03 | 0.57 ± 0.01 | 0.51 ± 0.02 |
| Other | 0.75 ± 0.00 | 0.89 ± 0.01 | 0.93 ± 0.00 | 0.95 ± 0.01 | 0.95 ± 0.00 |
| Binary Accuracy | 84.7% ± 0.00 | 95.6% ± 0.00 | 95.2% ± 0.01 | 95.6% ± 0.00 | 94.9% ± 0.00 |

* “Turn-level” represents the initial turn-level model trained with the coarse heuristic-based pseudo-labels.
* Accuracy is on the four functional classes only

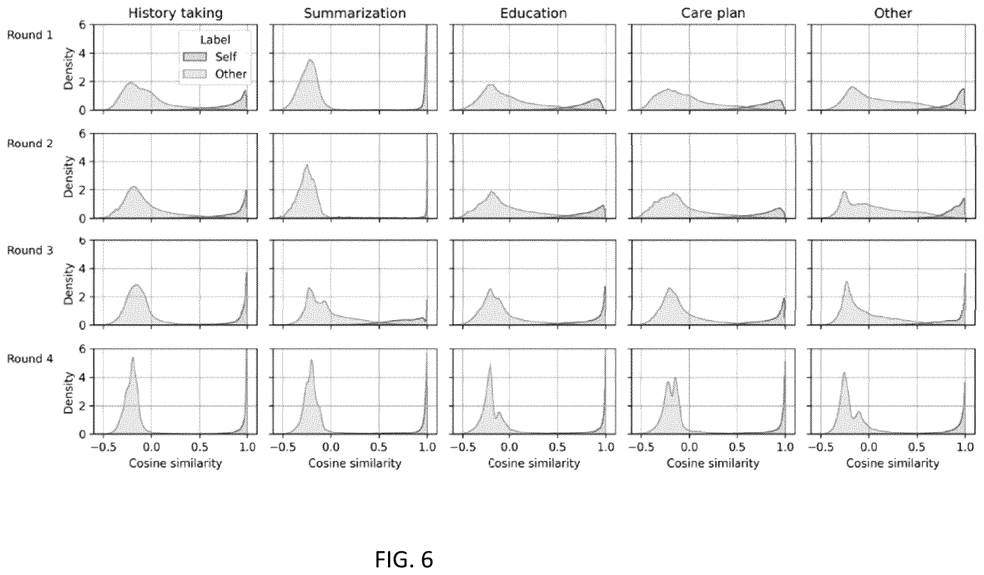

FIG. 6 sheds light on another perspective on the change in the quality of the latent space of the sentence-level models. Here, we randomly sampled 1,000 embeddings for each predicted class at each round and used them to compute the distribution of cosine-similarities between pairs of the same class (“self”) and pairs of different classes (“other”). Even for classes where the F1 metrics did not improve, there is a significant increase in the “peakiness” of the two distributions, making them more separable. This is the separation between positive and negative contrastive learning examples, where recent literature on sentence embeddings suggests that the increased separation corresponds to better generalization performance.

In the previous experiments, we evaluated the output of the sentence level model for each sentence in the input. Here, we investigate if training models at the sentence level also improve turn-level performance. We do this by pooling the predictions of all the sentences in a turn. For comparison, we use the initial turn level model as the baseline.

Table 3 shows the F1 and accuracy scores of the sentence-aggregated turn-level predictions. Like the sentence-level models, we see the most marked improvement in the “Summarization” section. Note how the Round 1 sentence-level model outperforms the turn-level model even though the turn-level model is used to generate the sentence-level pseudo-labels at the beginning with no human relabeling. This shows that the sentence-level model can learn semantics better and help drive model prediction.

Improvement from the later rounds is less pronounced when inferencing at the turn level. While sentence-level evaluation benefits from multiple rounds of disentangling the class confusion between sentences within a turn, this is less of a concern for turn-level evaluation. This is also evidenced by overall higher F1 scores when compared to evaluation at the sentence level in Table 2. However, since the same turn can have multiple labels, we report per-class binary accuracy.

### Auto-Charting Component 120

Example embodiments of the component 120 are based on the premise that specific rare tokens (e.g. metformin) have a high probability of appearing in a reference sequence if they also appear in the source sequence. Examples embodiments can determine which rare tokens have a propensity to appear in both source and target and how to encode this information into a machine learning model.

The example embodiments leverage knowledge that is outside the training set. This approach is likely to generalize to most high-stakes application domains since these domains are also ones that are knowledge-rich. In healthcare, there are ontologies such as UMLS and the International Classification of Diseases, that codify the medical terms and their relationships. Similarly, there are well-constructed ontologies in other domains including), e-commerce; and education.

Example embodiments are applied in the context of a healthcare setting. It is well-known that electronic health record charting is a significant source of medical professional burnout. The burden of the medical professionals can be significantly reduced by using Machine Learning (ML) systems that support charting by extracting most information for charting from medical professional-patient dialogue.

Accordingly, example embodiments generate care plan instructions from a medical dialog between patient and medical professional. We define the medical concept utilization rate and utilization-rate-aware training, discuss the care plan generation problem and data collection, describe the sequence-to-sequence model setup, and report experiments results. The proposed utilization rate quantifies the problem of rare concepts underestimation and can be effectively minimized during training through the soft marginal probability proxy. We observe performance improvement both for automatic and human evaluation with medical experts.

In many sequence-to-sequence tasks, certain rare concepts have a high probability to appear in the reference sequence (y) if they also appear in the source sequence (x). We call these concepts “high utilization concepts” (c∈C_{HU}) and formally define them in Equation 1. These concepts are comprised of one or more tokens c=[v₀, v₁, . . . ]. A source of factuality errors in many sequence-to-sequence tasks is that the learned model underestimates the conditional probability of high utilization concepts \hat{p}(yᵢ=v|y_{<i}, x, v∈c, c∈x, c ∈C_{HU})<p( . . . ), where p denotes the model estimated probability and p is the true probability.

Definition 2.1 (High utilization concepts). Given a universe of concepts C, the set of high utilization concepts C_{HU} is defined as

$${C}_{HU} = \{ c ∈ C : \frac{p ( p ∈ y \vert c ∈ x )}{p ( c ∈ y )} >> 1 \} \qquad ( 1 )$$

Therefore, example embodiments include:
1. a method for identifying high utilization concepts, C_{HU} for dataset [symbol] ={(xⁱ, yⁱ)}Nᵢ₌₁^{N}.
2. a method for augmenting the training procedure of seq2seq models to correctly estimate the conditional probability of tokens forming high utilization concepts.

### Identifying High Utilization Concepts Using Externally Provided Knowledge

A complication in identifying high utilization concepts in real datasets is that the concepts we are interested in are present in very few examples. This means that it is hard to directly estimate p(c∈y|c∈x) and p(c∈y) from Equation 1. However, these rare concepts can still be very impactful to the overall performance of the model. This is because, for a given reference, y, it is unlikely that a particular high utilization concept will be present (∀c∈C_{HU}, p(c∈y)«1), but it is also unlikely that no high utilization concept will be present Π_{c∈C}_{_{HU}} p(c∈y).

To overcome this challenge, example embodiments compute a “utilization rate”, r_{ϕ}, which we define in Equation 2. This function relies on the concept equivalence class map ϕ: Cₛₑₗ→ε where Cₛₑₗ⊆C and ε is a set of equivalence classes. (ϕ, Cₛₑₗ, ε) cannot be derived from the data or the model, but instead are provided from an external source of knowledge. If ϕ is an identity (id) then r_{id}(cₙ)=\hat{p}(cₙ ∈y|cₙ ∈x), (x, y)∈D.

Definition 2.2 (Utilization rate). The utilization rate of concept cₙ is defined as

$${r}_{ϕ} ( {c}_{n} ) = \frac{{\sum }_{c ∈ {C}_{sel}} {\sum }_{j}^{N} I [ c ∈ {x}^{j} , c ∈ {y}^{j} , ϕ ( c ) = ϕ ( {c}_{n} ) ]}{{\sum }_{c ∈ {c}_{sel}} {\sum }_{j = 1}^{N} I [ c ∈ {x}^{j} , ϕ ( c ) = ϕ ( {c}_{n} ) ]} \qquad ( 2 )$$

Note that Equation 2 combines both externally provided knowledge (ϕ, Cₛₑₗ, ε) and dataset derived values. This allows us to inject domain-specific information. Because concepts are mapped to equivalence classes, every concept in a particular equivalence class has the same utilization rate. If a concept cₙ∈Cₛₑₗ has marginal probability to appear in the reference sequence that is much lower than r_{ϕ}(cₙ) then it is a high utilization concept.

Utilization-Rate-Aware seq2seg Training

Per the analysis above conventionally trained seq2seq models underestimate the utilization rate (r_{ϕ}) for many rare concepts. While we cannot optimize the utilization rate directly, we can optimize the approximate marginal probability p(v|x) of a token v given a source sequence x, as seen in Equation 3.

$$p ( v \vert x ) = {\sum }_{{y}_{< t}} p ( v \vert {y}_{< t} ) p ( {y}_{< t} ) \approx {\sum }_{t = 1}^{‖ y ‖} p ( v \vert {y}_{< t} ) p ( {y}_{< t} ) \approx \frac{1}{‖ y ‖} {\sum }_{t = 1}^{‖ y ‖} p ( v \vert {y}_{< t} ) \qquad ( 3 )$$

Given the source sequence x, the tokens for which we aim to optimize the marginal probability are (v∈c, c∈x∩C_{HU}). We define the unweighted utilization loss.

Definition 2.3 (Unweighted utilization loss).

$${l}_{u} ( x ) = - \frac{1}{‖ \{ v ∈ c , c ∈ x ⋂ {C}_{HU} \} ‖} {\sum }_{v ∈ c , c ∈ ( x ⋂ {C}_{HU} )} log p ( v \vert x ) \qquad ( 4 )$$

However, not all concepts in C_{HU} are equally likely to appear in the reference given their appearance in the source. To better reflect we also propose a weighted utilization loss where the weight for each token is determined by its utilization rate.

Definition 2.4 (Weighted utilization loss).

$${l}_{w} ( x ) = - \frac{{\sum }_{v ∈ c , c ∈ ( x ⋂ {C}_{HU} )} {r}_{ϕ} ( c ) log p ( v \vert x )}{{\sum }_{v ∈ c , c ∈ ( x ⋂ {C}_{HU} )} {r}_{ϕ} ( c )} \qquad ( 5 )$$

Note that Equation 5 directly injects externally provided knowledge through its dependence on ϕ.

We use utilization loss as a regularization term and augment the objective function. We use α>0 to balance the strength of the regularization:

l(x,y)=lₙₗₗ(y)+α·l_{u or w}(x) (6)

where lₙₗₗ=−Σₜ₌₁^{|y|} log p(yₜ|y_{<t}, x) and l_{u or w} is either lᵤ from Equation 4 or l_{w} from equation 5.

### Care Plan Instruction Generation: Task and Data Description

A care plan is a set of actions (instructions) that a medical professional writes in the patient's electronic health record (EHR) as a follow-up to the patient's visit. A care plan often includes a list of medications with appropriate directions, further medical evaluations, or educational information for preventive care. Before writing the care plan, the medical professional discusses it with the patient, and together, they jointly agree on the next course of action. This joint decision-making implies that most of the necessary information for writing the care plan is already available in the conversation.

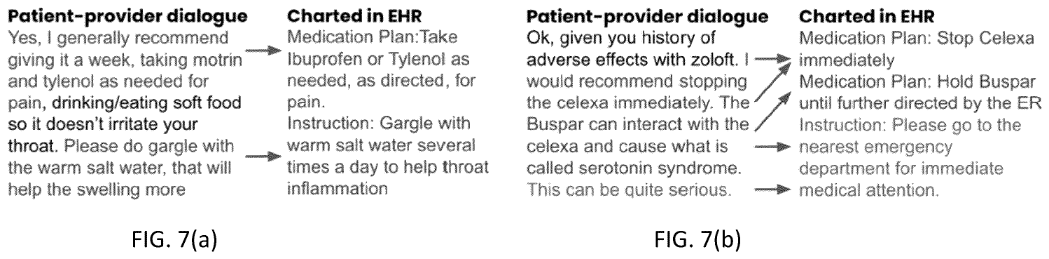

In FIG. 7(a) and FIG. 7(b) illustrate two examples. FIG. 7(a), illustrates a relatively simple-to-chart example with each sentence corresponding to an instruction. Note synonym substitution of ibuprofen for motrin and the addition of timing to the gargling instruction. In contrast, FIG. 7(b) illustrates a difficult-to-chart example with incomplete information and multiple dialogue sentences contributing to a single instruction.

In each example, there is (a) segment of the conversational dialog corresponding to provider messages discussing the care plan with the patient and (b) corresponding care plan charted in the EHR Instructions are written in a directive format, using action verbs and often paraphrasings of the corresponding text in the dialog. The care plan does not always have all the medical concepts mentioned in the conversation. In the first example, ‘serotonin syndrome’ and ‘Celexa’ are rare, but the care plan includes only the latter. We need a model that is robust to rare medical concepts and can discern which knowledge needs to be carried forward.

Example embodiments take the relevant section in the conversations corresponding to the care plan as input and automatically derive care plan instructions that the medical professionals can approve. There may not be access to 1-1 mappings between the sentences in the conversation to the care plan instructions. However, example embodiments provide a method to derive a dataset of 1-1 mappings, albeit noisy, which can be used for model training.

Dataset construction. The dataset may comprise 14K medical professional-patient encounters collected on a virtual primary care platform. Each encounter has a text-based conversation between the medical professional and the patient. A conversation discourse parser extracts only those dialogue turns from the medical professional's corresponding to care plan discussion. Associated care plans written from the patient's electronic health record for that encounter can also be used. On average, each encounter has 9 dialogue turns corresponding to care plans and 4 care plan instructions.

A parallel corpus with pairs of dialogue turn and care plan instruction for our model are needed. Getting manual annotations for each encounter would be expensive as it requires expert knowledge. Therefore, example embodiments automatically construct a paired dataset, albeit noisily, from the paired encounter level care plan and provider dialog turns. We get sentence-level embeddings for every sentence in each turn and instructions in the care plan and pair those with the highest cosine similarity. At the end of this, we have 48,000 pairs of source-reference pairs, where the source is a sentence in the conversational dialog and reference is the reference pair mapped for the instruction. We randomly sample 3000 pairs for testing, 1000 for validation, and remaining 44,000 pairs for training.

To identify the concepts, we use a lookup-based concept recognizer. It uses a sliding window strategy to find maximal matches of text corresponding to medical concepts and their synonyms. It ignores stop words while doing the match. We use medical concepts from UMLS and in particular SNOMED-CT and RXNorm ontologies. The synonyms are pooled from all ontologies in UMLS that maps to corresponding concept in SNOMED-CT and RXNorm.

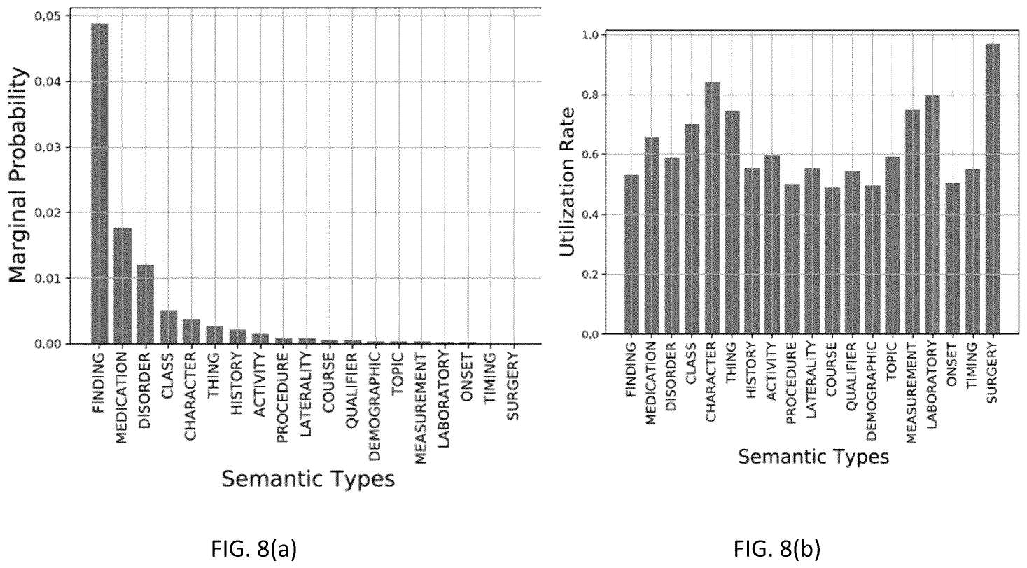

Identifying high utilization concepts. We limit Cₛₑₗ to only medical concepts and choose ϕ such that it maps them to their SNOMED CT semantic types (which informs our choice of ε). In our case study this narrows down 758 unique medical concepts to their 19 semantic types. The marginal probability for each semantic type is shown in FIG. 8(a) while the utilization rates are shown in FIG. 8(b). Comparing them we can see that utilization rates are 10-100× larger than the marginal probabilities. Therefore, all medical concepts are part of high utilization tokens set (C_{HU}=Cₛₑₗ). It also means that many kinds of medical concepts that are present in the source sequence do not get generated in the output sequence, which drastically hurts medical correctness.

### Experimental Setup

We follow the standard practice (Ott et al., 2018) of training our sequence-to-sequence models using FairSeq framework (Ott et al., 2019). We use byte-pair encoding implemented in the fastBPE package ((Sennrich et al., 2016)). We use a transformer architecture for our model and train models on our data from scratch.

Model architecture We use the transformer_iwslt_de_en architecture in FairSeq for experiments. It comprises 6 encoder and decoder layers with 4 self-attention heads followed by feed-forward transformations. Both encoder and decoder use embeddings of size 512 while the input and output embeddings are not shared. Both the encoder and decoder use learned positional embedding. We early-stop training based on the validation performance. Evaluation is done on the test set.

Training We use Adam optimizer with β₁=0.9 and β₂=0.98. We use the inverse square root learning scheduler with 4,000 warm-up steps. We use the initial learning 188 rate of 5×10⁻⁴, dropout rate of 0.3, and weight decay with its rate set to 10⁻⁴. We use label smoothing with 0.1 of probability smoothed uniformly during training. We modify the training objective Equation 6 by adding oversmoothing loss with a coefficient of 0.9 and unlikelihood loss with a coefficient of 0.5. All training was performed on VMs with single V100 GPUs, we estimate 200 GPU hours as the total amount required for the completion of this work.

Early stopping We use early stopping for model selection based on the value of the objective function computed on the validation set. We evaluate the model on the development set every 2K updates (˜4K tokens per update). We stop training when the objective has not improved over more than 5 consecutive validation runs. It takes approximately 75K updates to an early stop.

Decoding We use beam search implementation from FairSeq. We decode using the beam size of 5. We set the lower- and upper-bound of a generated output to be, respectively, 0 and 1.2·∥x∥+10. We do not use either length normalization or length penalty since we apply oversmoothing loss.

Lexically constrained decoding baseline Apart from using the unregularized version of the model as a baseline, we compare the proposed approach with the lexically constrained decoding approach. We stick to the LexicallyConstrainedBeamSearch implementation of the Dynamic Beam Allocation (DBA) algorithm that ensures the presence of provided tokens in the generated output. DBA implements an optimized version of the Grid Beam Search. DBA is training-agnostic and is used only during generation. We apply DBA for the baseline model. Given the non-uniform distribution of utilization rates, for each source we leave only medical concepts c with r_{id}(c)>τ for some threshold τ. We report results for τ=0.6, which we select by running an extensive grid search.

### Results

### Effect of Knowledge Injection During Training on Model's Utilization Rate Estimation

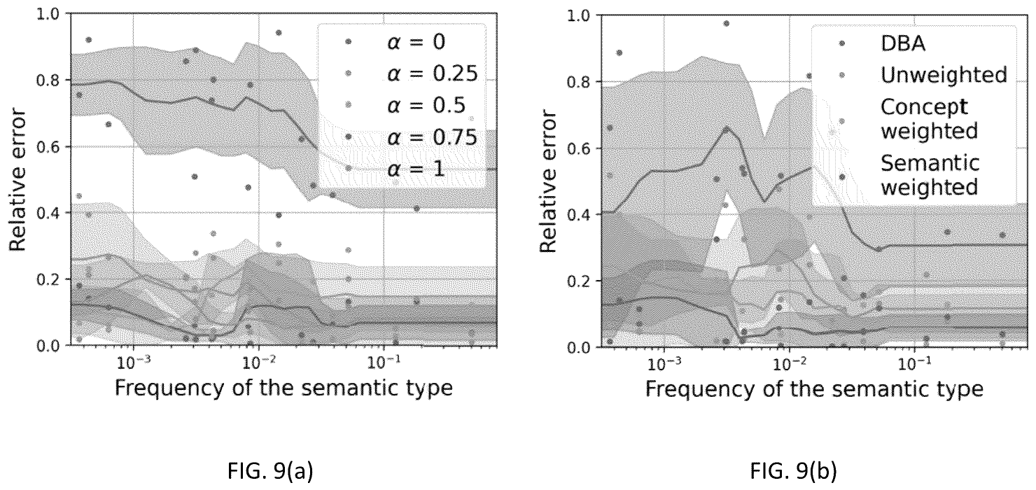

We evaluate whether the knowledge injection through regularization has the desired effect of improving model estimate of the utilization rate, rf. Because the test set is too small to effectively estimate per-concept utilization rate, we instead compute it for semantic types. In FIG. 9(a) and FIG. 9(b) we use semantic relative error (Equation 7) to compare models trained with α∈{0, 0.25, 0.5, 0.75, 1} that either use unweighted loss lᵤ (which uplifts all medical concepts equally, “Unweighted”) or a weighted loss l_{w} with the ϕ being identity (“Concept weighted”) or mapping concepts to semantic types (“Semantic weighted”). In addition, as a baseline we also compare an unregularized model that uses DBA for generation (“DBA”). For a detailed breakdown of relative errors for each combination see the Supplementary Material.

Definition 5.1 (Semantic relative error). Relative error for semantic type s computed from r_{ϕ} estimated from model derived output sequences and r-estimated from reference sequences. cₛ is any concept for which ϕ(c)=s holds and the value of ∈ₛ in not dependent on the choice of cₛ.

$${ϵ}_{s} = \frac{‖ {r}_{ϕ} ( {c}_{s} ) - {r}_{ϕ} ( {c}_{s} ) ‖}{{r}_{ϕ} ( {c}_{s} )} \qquad ( 7 )$$

In FIG. 9(a) we present the relative error for different α as a function of semantic type frequency in the test set. For each point (a given semantic type and α) we take the lowest relative error among (“Unweighted”, “Concept weighted”, and “Semantic weighted”). The highest relative errors are seen for α=0, which corresponds to no regularization. For other values of α the difference is not statistically significant, although for very rare semantic types, α=0.25 appears to perform worse than models with higher regularization strength. This shows that our external knowledge informed regularization has a significant impact on a relative error, but the utilization rate estimate is not sensitive to the exact weight of the regularization term.

In FIG. 9(b) we present relative error for different training procedures, (“Unweighted”, “Concept weighted”, and “Semantic weighted”), as well as a baseline of “DBA.” For each point (a given semantic type and training procedure) we choose an a that gives the lowest relative error. We find that “DBA” baseline, which is a constrained generation procedure applied to an unregularized model, performs worse than any of the regularized models, although it does outperform the unregularized model ((x=0 in FIG. 9a). While not significant, we also see that for rare semantic types “Semantic weighted” seems to perform the best, which aligns with our expectation that the utilization rate is hard to estimate for very rare concepts.

### Effect of Knowledge Injection During Training on Model's Uncertainty

We analyze the effect of utilization regularization on the model's uncertainty at every timestep. Uncertainty at timestep t is defined as an entropy of model's distribution on each timestep t:

Hₜ(y,x)=−Σ_{y}p(y|y_{<t},x)log p(y|y_{<t},x) (8)

We consider the defined uncertainty on earlier timesteps, where the model's distribution is closer to marginal. As the proposed method pushes up the marginal probability of the medical concepts, we claim that models' uncertainty decreases with the regularization. Moreover, care plan instructions typically introduce crucial concepts at the beginning of an instruction. Thus, we claim that early timesteps uncertainty matters for the precise decoding of instructions.

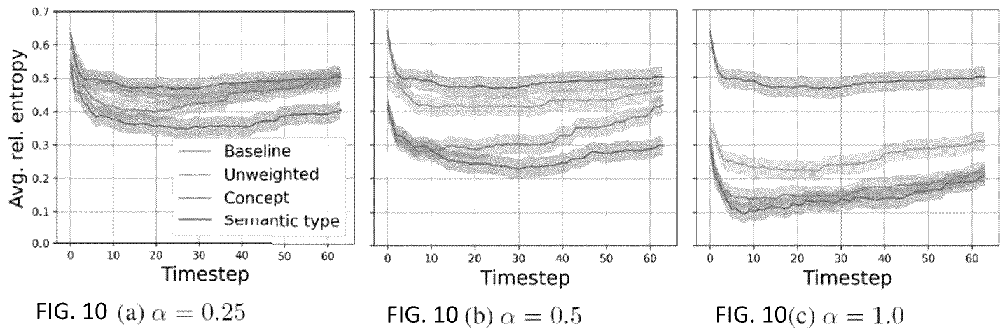

This is confirmed by FIGS. 10(a), (b) and (c). We observe that uncertainty drops monotonically as the a weight increases. In particular, uncertainty on early timesteps heavily drops as a result of utilization minimization. Hence, the model becomes more confident in selecting principal concepts at the beginning of an instruction. In contrast to the baseline, all regularized models' uncertainty stats to increase for t>10. As fewer concepts appear in the instruction end, the marginal probability maximization flattens the conditional distribution. However, the uncertainty does not degrade in comparison to the baseline. Thus, the proposed regularization effectively improves the confidence of the model on early timesteps.

### Results on Care Plan Instructions Task

TABLE 4
Automated metrics scores for different model setups. We report
average score and standard deviation over five random seeds.
We highlight in bold the best average and all scores having
overlapped standard deviation intervals with the best score.

| | | | | GPT-2 |
|---|---|---|---|---|
| | α | BERTScore | Concept-F1 | Perplexity |
| Baseline | 0.0 | 22.48 ± 0.66 | 57.43 ± 3.73 | 5.53 ± 0.04 |
| DBA | — | 23.59 ± 0.28 | 79.83 ± 0.43 | 11.96 ± 0.05 |
| Unweighted (ours) | 0.25 | 25.09 ± 0.69 | 58.19 ± 2.11 | 5.91 ± 0.07 |
| | 0.5 | 25.42 ± 0.56 | 58.91 ± 6.83 | 5.65 ± 0.03 |
| | 0.75 | 26.22 ± 0.35 | 60.83 ± 5.96 | 6.28 ± 0.02 |
| | 1.0 | 26.74 ± 0.43 | 61.05 ± 7.48 | 6.18 ± 0.05 |
| Concept weighted | 0.25 | 28.29 ± 0.19 | 60.87 ± 3.86 | 6.93 ± 0.05 |
| (ours) | 0.5 | 28.19 ± 0.20 | 60.36 ± 2.03 | 8.49 ± 0.05 |
| | 0.75 | 28.08 ± 0.15 | 64.09 ± 1.85 | 7.95 ± 0.080 |
| | 1.0 | 27.82 ± 0.25 | 63.05 ± 2.49 | 9.37 ± 0.10 |
| Semantic weighted | 0.25 | 28.97 ± 0.56 | 69.10 ± 2.12 | 7.01 ± 0.29 |
| (ours) | 0.5 | 30.54 ± 0.78 | 74.98 ± 3.91 | 6.84 ± 0.03 |
| | 0.75 | 31.48 ± 0.86 | 75.77 ± 3.30 | 6.96 ± 0.11 |
| | 1.0 | 30.59 ± 0.63 | 75.02 ± 2.18 | 6.94 ± 0.12 |

Automated evaluation: The precise and complete concepts utilization directly affects the quality of instruction. We first quantify the quality by calculating automatic metrics to judge the relevance, fluency, and concept utilization rate in comparison to the reference instructions. We use BERTScore to estimate the similarity between reference and candidate, GPT-2 perplexity for to assess the coherence (fluency) of the candidate, and concept overlap to measure the percentage of medical concepts used in both candidate in reference.

Table 4 presents the automatic evaluation results. The scores indicate that incorporating knowledge correlates with relevance and concept overlap. We highlight three observations. First, the regularization is effective in terms of quality and concept overlap. We observe significant quality improvement compared to both the baseline and DBA. Moreover, weighted versions of the model outperform the unweighted setup. Thus, injecting more knowledge into the model, such as empirical utilization weights, results in better quality. Second, the impact of the regularization hardly depends on the a weight Third, the GPT-2 perplexity degrades. This demonstrates that the regularization impacts the model distribution, so the fluency of the model may deteriorate. This trade-off, however, has no negative impact on the quality. For qualitative results, please see the Supplementary Material.

Medical experts evaluation: To get a more precise medical assessment, we conduct human evaluation with medical experts. We randomly sample 100 dialogues from the test set and generate candidates with each model setup setting α=1.0. We ask five doctors to evaluate the relevance to the dialogue, medical usability (if the generated instruction can be used in any care plan), and grammatical correctness (fluency) on a scale from 1 to 5. Additionally, we ask assessors to indicate degenerate generations, i.e., premature or repetitive sequences. Exact questions and interface screenshots can be found in the Supplementary Material.

As shown in Table 5, we claim that both weighted versions achieve significant improvement in relevance and usability, which are target medical metrics. In contrast to the GPT-2 perplexity, medical experts report equal fluency for all models but DBA. We explain this discrepancy with vocabulary shift as GPT-2 is not trained on a healthcare corpus. Finally, utilization rate regularization does not affect the number of degenerate outputs. Hence, the proposed solution effectively induces knowledge in the model distribution without corrupting generated text correctness. This is not true for DBA, which struggles from a lack of coherence and degenerate outputs while producing more relevant and usable instructions.

TABLE 5
Evaluation using medical experts. Fluency, Usability, and Relevance are scored
on a scale from 1 to 5. We also report the percentage of premature or repetitive
outputs (Degeneracies). We report average score and standard deviation of
experts' scores. We highlight in bold the best average and all scores
having overlapped standard deviation intervals with the best score.

| | Relevance | Usability | Fluency | Degeneracies, % |
|---|---|---|---|---|
| Baseline | 2.50 ± 0.12 | 3.18 ± 0.27 | 4.17 ± 0.14 | 0.10 ± 0.01 |
| DBA | 3.36 ± 0.15 | 3.35 ± 0.16 | 3.91 ± 0.18 | 0.21 ± 0.05 |
| Unweighted (ours) | 3.56 ± 0.12 | 3.21 ± 0.28 | 4.26 ± 0.08 | 0.10 ± 0.02 |
| Concept weighted | 3.79 ± 0.06 | 3.72 ± 0.05 | 4.37 ± 0.16 | 0.12 ± 0.02 |
| (ours) | | | | |
| Semantic weighted | 3.78 ± 0.14 | 3.99 ± 0.19 | 4.42 ± 0.13 | 0.12 ± 0.012 |
| (ours) | | | | |

Conclusion

In this work, we tackle the problem of under-generation of rare but important tokens in sequence-to-sequence models. We show that external knowledge can be effectively injected into the sequence-to-sequence models and mitigate the problem of lexical precision. We characterize the problem by identifying a set of low-frequency but important concepts and defining their utilization rate, which estimates the probability of a concept presented in the source to also be in the reference. We confirm that modem well-trained sequence-to-sequence models suffer from under-estimating utilization rates, and propose a way to directly maximize it during training. We design a differentiable proxy based on the marginal entropy and suggest a regularized training objective. Since some concepts may be omitted from the reference, we extend the approach by applying weights, which restrict the regularization impact of low-utilized concepts or their semantic types.

We perform a case study in automatic care plan generation from medical dialogues. We experiment with a custom internal dataset and observe the effectiveness of the approach. We also compare to a previous approach for external knowledge injection—dynamic beam allocation (DBA). First, we find that regularization improves the model's utilization rate by pushing it closer to the empirical values observed in reference sequences. Second, regularization reduces the model's uncertainty at early timesteps: exactly where concepts are typically introduced. Third, we observed a significant (in terms of standard deviations) quality improvement More specifically, we did a human evaluation of relevance, concept overlap, medical usability, and fluency using five medical experts. The results revealed the enhanced relevance and usability of generated instructions while, unlike DBA, maintaining high fluency and low degeneracy.

### Summarization Component 130

Example embodiments provide a medically-aware the summarization component 130, e.g., a Machine Learning (ML) model data labeler, GPT-3-ENS, that combines medical knowledge and an ensemble of GPT-3 for the purpose of medical dialogue summarization. While GPT-3 is used in an example, other machine learning models (large language models) may be used.

Example embodiments use GPT-3-ENS as a dataset generator to facilitate learning an in-house summarization model. Our experiments show that we can obtain the same performance as that of human labeled dataset with 30× smaller amount of human labeled data. With only 210 expert curated summaries and GPT-3 as a labeled data simulator, we can mimic the performance of a summarization model trained on 6400 expert curated summaries.

By combining generated datasets from GPT-3-ENS with a human labeled dataset, we show that we can obtain better performance than models trained on either one of the data sources.

One of the main challenges in using deep learning for healthcare is the lack of large annotated datasets. It is usually costly and time-consuming to collect a large labeled dataset because annotations need to be provided by trained healthcare professionals. As deep models usually require a large amount of data to perform accurately and robustly, this deters their widespread application in healthcare. So, it is essential to develop low-shot models in healthcare i.e. models that can do well given a small number of labeled examples. In parallel, there has been a lot of progress in development of large scale models leveraging web-scale data, such as GPT-3, that show good low-shot performance. However, these models can be noisy, particularly in the medical domain, so we need approaches that mitigate this noise but are still able to leverage these models' strengths. In this context, our approach of infusing medical knowledge in pretrained models such as GPT-3 to generate high-quality synthetic labels is an idea with wide applicability in low-resource settings like healthcare.

If pretrained models can be used to generate accurate labels, can they be directly leveraged for the task at hand? In many settings, they probably can but particularly in healthcare, this is nuanced. ML models in healthcare can learn and improve over time only if they are amenable to feedback loops i.e., they can be retrained with labels that are corrected/edited by medical practitioners. Moreover, if the model making the predictions is owned by a third party, privacy protocols (e.g. HIPAA) mandate that either they obey the same privacy protocols or that data be deidentified before being sent to such external services. Both these necessitate the need for a different approach. Accordingly, example embodiments infuse medical knowledge into an external non-HIPAA compliant model (GPT-3) and leverage it as a data generator to obtain a large training set, to then train an in-house model. Since the data exposed to GPT-3 is fixed and small (in our experiments, GPT-3 only saw 210 examples), it can be ensured to be privacy protected. Our proposed approach to develop an in-house model has two advantages (1) It can be used at inference time without the practical constraint of data de-identification and (2) It lends itself well to the aforementioned practitioner-in-the-loop setting.

### Infusing Medical Knowledge in GPT-3 for Use as a Data Generator

We are interested in a model that uses only a small amount of human labeled data to learn an effective medical dialogue summarizer. At the same time, we want such a model to be used in a practical practitioner-in-the-loop setting where medical correctness and patient privacy are of paramount importance.

In order to achieve these goals, example embodiments:
1. Introduce GPT-3-ENS where we infuse medical knowledge into GPT-3 and use it within an inner loop to make it effective at medical summarization.
2. Leverage GPT-3-ENS as a data generator to obtain a large training set to train an medical dialogue summarization model. Such a model can be used at inference time without the practical constraints related to protecting patient privacy that would require full de-identification to be applied in any conversation, if we were to access the GPT-3 service. It also lends itself well to the practitioner-in-the-loop setting.

### GPT-3-ENS: Medically-Aware Ensemble of GPT-3

As discussed above, GPT-3 is quite sensitive to the priming context. While one approach may be to provide GPT-3 with the most informative context for a task, this itself is a daunting task and can potentially be tackled if we had a large number of labeled examples (which is the exact problem we want to tackle with GPT-3).

If we can generate multiple summaries from GPT-3 using a variety of priming contexts, then we should be able to ensemble these outputs to identify the summary that is ideal for the dialogue. This insight leads to a question on how to ensemble multiple text summaries. The answer to this question relies on the core requirement for medical summarization: we care about the coverage of medical concepts mentioned and therefore the best ensembling function is the one that returns the summary with the most medical information in the dialog input.

TABLE 6
Input dialogue snippets along with summaries generated by
GPT-3 in column 2 and our approach, GPT-3-ENS, in column 3.

| Snippet | GPT-3 | GPT-3-ENS |
|---|---|---|
| DR: Thank you so much | On birth control. | Only regular medication |
| for sharing. Are you on | | is birth control -Apri. |
| any regular medications | | Low dosage. |
| for that? | | |
| PT: My only regular | | |
| medication is birth control | | |
| -Apri. Low dosage. | | |
| DR: You had mentioned | Yes. Stopping | Yes, has headache while |
| the headache starting after | medications before. | stopping medications |
| stopping the medications. | | before. Is not sure. |
| Have you had similar | | |
| headache while stopping | | |
| medications before? | | |
| PT: Yes | | |
| PT: Well that's a | | |
| complicated question | | |
| PT: I'm not really sure | | |
| DR: Okay, no worries. | | |
| Wanted to know a bit | | |
| more since you had | | |
| mentioned about them. | | |
| That's all. | | |
| DR: Do you have pain | Did not notice penile | Doesn't have pain when |
| when you notice penile | discharge. No pain. | noticing penile discharge. |
| discharge? | | |
| PT: no i'm not | | |
| DR: I have a few | I have a few questions to | Has been having vaginal |
| questions to ask. How | ask. How long has she | discomfort for only a few |
| long have you been | been having vaginal | days, since Friday or |
| having this vaginal | discomfort? | Saturday. |
| discomfort? | | |
| PT: only a few days, since | | |
| like Friday or Saturday | | |
| maybe | | |

In Algorithm 1 we provide our approach to the medically aware GPT-3 ensemble GPT-3-ENS. We assume access to a small set of labeled examples [symbol] . For each input dialog snippet, T, we get K summaries, by invoking GPT-3 each time with N examples sampled randomly without replacement from [symbol] . We also assume access to a medical entity extractor that can discern the medical concept from both the dialogue snippet and the summary. The algorithm returns the best summary that has the highest recall in terms of capturing the medical concepts in the dialogue. For this purpose, we use an in-house medical concept extractor MEDICALENTITYRECOGNIZER that can identify medical concepts from a given piece of text. This extractor has access to the universe of medical concepts based on Unified Medical Knowledge Systems, which includes patient symptoms, disorders, laboratory tests and medications. Note that any medical entity recognizer that has coverage for all these types of medical concepts found in medical conversations can be used.

Algorithm 1 Medically aware GPT-3 ensemble summarizer (GPT-3-ENS)
Require: dialogue snippet T, ensembling trials K, universe [symbol] of labeled
examples, medical entity extractor
MEDICALENTITYRECOGNIZER, GPT3

| | |
|---|---|
| 1: | C* ← MedicalEntityRecognizer(T) |
| 2: | for i ← 1, · · · , K do |
| 3: | S ← sample N examples from [symbol] |
| 4: | summaryᵢ ← GPTS(S,T) |
| 5: | Cᵢ ← MedicalEntityRecognizer(summaryᵢ) |
| 6: | ${summary}_{best} ← summary arg {max}_{i} \frac{\vert {C}_{i} ∩ {C}^{*} \vert}{\vert {C}^{*} \vert}$ |
| 7: | return summary_{best} |

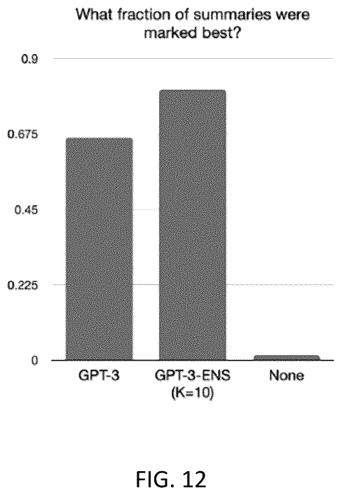

Reconsider Table 6 for qualitative comparison between GPT-3 and GPT-3-ENS. We can see that summaries obtained using GPT-3-ENS capture the medical concepts comprehensively (shown in bold) and also have better grammatical structure. We also quantitatively validate the summaries on a small data set distinct from what is used for priming(see § 7.2 for guidelines). In FIG. 12, based on doctor evaluation, we can see that GPT-3-ENS is significantly better at summarization than GPT-3.

### GPT-3-ENS as a Data Labeler

We use GPT-3-ENS as our labeled data generator. In particular, we use our approach to collect a large amount of labeled examples that serve as inputs to training an off-the-shelf summarization model. This resolves the concern of using GPT-3 in a real world application where the patient's conversation (in its raw form) needs to be exchanged with an external third party such as OpenAI/GPT-3 which may not have design/privacy regulations around HIPAA.

### Datasets

We collected a random subset of medical conversation dialogues from our chat-based telemedicine platform. Often medical conversation follows a linear ordering of medical history gathering (understanding patient symptoms) that enables creating the summary of the dialog by stitching together summaries of the snippets in chronological order. Therefore, we split each dialogue into a series of local dialogue snippets using a heuristic: the turns between two subsequent questions by a physician corresponds to a snippet. The length of these snippets ranged anywhere from two turns (a physician question and patient response) to ten turns.

We had medical doctors summarize these snippets. The doctors were asked to summarize the sections as they would for a typical clinical note by including all of the relevant history taking information. If a local snippet did not contain any history taking information it was excluded from annotations. For example in the beginning or end of conversations there may be turns that are purely greetings and not part of the patient history taking process. Further some snippets maybe purely educational in nature and are excluded as well. We eventually obtained a total of 6900 labeled snippet-summary pairs.

Human labeled dataset train/test split: From the 6900 labeled snippet-summary pairs (denoted as H₆₉₀₀), we generated a randomly sampled test set T=500 that we use in all our evaluations.

The dataset H₆₉₀₀-T is used to generate the priming dataset for GPT-3 related models as well as the datasets we use to train our summarization models.

GPT-3-ENS dataset: Let GCFₚ^{k} be the dataset of size p generated using GPT-3-ENS with k ensembling trials. To generate dataset GCF^{K=k}, we require {Hₙ}ᵢ₌₁^{k} datasets (note the independence on p), and thus n×k labeled examples for priming. These n×k examples are randomly sampled from the universe of human labeled examples H₆₉₀₀-T. In our experiments, we sample without replacement so that no examples are reused across the k tries. To allow comparison between our experiments with different K values, we use the same seed for random sampling.

Evaluation Metrics

Automated Metrics

While we measure model performance on standard metrics of, we also measure a model's effectiveness in capturing the medical concepts that are of importance, and their negations.

Medical Concept Coverage: The concept coverage set of metrics captures the coverage of medical terms in the model's output summary with respect to the ground truth. In particular, let C be the set of medical concepts in the reference summary and Ĉ be the set of concepts in the summary output by the model. Then

$$Concept recall = \frac{{\sum }_{n = 1}^{N} \vert {\hat{C}}^{( n )} ⋂ {C}^{( n )} \vert}{{\sum }_{n = 1}^{N} \vert {C}^{( n )} \vert} and Concept precision = \frac{{\sum }_{n = 1}^{N} \vert {\hat{C}}^{( n )} ⋂ {C}^{( n )} \vert}{{\sum }_{n = 1}^{N} \vert {\hat{C}}^{( n )} \vert} .$$

We use these to compute a Concept F1. We use a medical entity extractor to extract medical concepts in the summary. Medical concepts in the decoded summary that weren't present in the original conversation would be false positives and vice versa for false negatives.

Negation Correctness: Of the concepts present in the decoded summary, we evaluate precision and recall on whether the decoded negations were accurate for the decoded concepts and compute a negation F1.

Doctor Evaluation

We also had doctors evaluate the summaries produced by the models. Given the local dialogue snippets and the generated summary, we asked them to evaluate the extent to which the summary captured factually correct and medically relevant information from the snippet Depending on what percentage of the concepts were correctly mentioned in the decoded summary of the provided snippet, the doctors graded the summaries with All (100%), Most (at least 75%), Some (at least 1 fact but less than 75%), None (0%) labels.

We also formulated a comparison task where given summaries generated by different models and the associated dialogue, they were asked which summary was the “best” from a usability perspective. Usability was defined as whether the summary could stand in as a replacement for reading the dialogue snippet i.e. whether it captures the correct concepts from the snippet and whether the negations are accurate. The doctors had the ability to use “all” and “none” in this task depending on if all models being compared captured a good summary or if none of them did.

To avoid bias, the doctors do not know the model that produced the summary in both the experiments. In the comparison task, the summaries were provided in randomized order so that there is no bias in the order of presentation of the summaries.

Experiments and Results

Implementation Details: We used GPT-3 via the API released by OpenAI. Maximum response length was set to 128 tokens, temperature to 0.6 and presence and frequency penalties both set to 0. For GPT-3-ENS, we use K=10 ensembling trials for all our experiments, unless otherwise specified. We observed that N=21 was the maximum number of examples we could prime GPT-3 with given the maximum context window length of 2048 tokens for the API. We therefore fix the size of our priming dataset to be 21 in all experiments which invoke GPT-3. Hence we set L to be a random subset of 210 examples from H₆₉₀₀-T.

We followed parameter settings for DRSUM from Joshi et al. (2020) for pretraining on the CNN-Dailymail dataset. We then fine-tuned on our summarization task dataset with a batch size of 16, source_max_tokens=400, response_max_tokens=200 and max_grad_norm clipped at 2.0, for two epochs with a learning rate of 0.15 using Adagrad optimizer.

We used the PEGASUS implementation that is pretrained on CNN-Dailymail. We fine-tuned it on our summarization task dataset with an effective batch size of 256, source_max_tokens=512, response_max_tokens=128 for two epochs using Adafactor optimizer at the default settings in Hugging Face. For both PEGASUS and DRSUM, we used a beam size of four for decoding.

8.1. Training Summarization Models Using Data Labeled by GPT-3-ENS

We compare PEGASUS and DRSUM trained on human labeled data H₆₄₀₀ and GPT-3-ENS synthesized data GCF₆₄₀₀^{K=10}. Note that synthesizing GCF₆₄₀₀^{K=10} needed all of 21·10=210 human labeled examples, where 21, as a reminder, is the maximum number of inputs that can be used for priming.

Table 7 compares quantitative performance of PEGASUS and DRSUM trained on these two datasets. The main observation is that with only 210 human labeled examples, our approach GPT-3-ENS is able to generate a large amount of training data for both pre-trained summarization models, PEGASUS and DRSUM, in such a manner that they yield comparable (or better performance) than if they had been trained with only 6400(˜30×) human labeled examples.

TABLE 7
Automated evaluation of summarization models trained with
different data labeling methodologies. Note that the
amount of human labeled data is still pretty low (210),
compared to 6400 when we do not use our approach.

| | | Metrics | | |
|---|---|---|---|---|
| | Train Data | Negation | Concept | ROUGE-L |
| Models | Source | F1 | F1 | F1 |
| PEGASUS | H₆₄₀₀ | 21.09 | 35.96 | 55.59 |
| | GCF₆₄₀₀^{k=10} | 28.89 | 40.02 | 53.43 |
| | GCF₁₂₈₀₀^{k=10} | 26.70 | 40.21 | 56.66 |
| | GCF₂₅₆₀₀^{k=10} | 28.61 | 40.58 | 58.44 |
| DRSUM | H₆₄₀₀ | 26.75 | 39.95 | 52.70 |
| | GCF₆₄₀₀^{k=10} | 24.29 | 37.55 | 48.47 |
| | GCF₁₂₈₀₀^{k=10} | 26.66 | 38.49 | 49.18 |
| | GCF₂₅₆₀₀^{k=10} | 26.08 | 39.47 | 50.85 |

For PEGASUS, the summarization performance improves drastically compared to model fine-tuned using only the human labeled data. We hypothesize that data generated from GPT-3-ENS can serve as quality training data for abstractive models such as PEGASUS but not so much for hybrid models such as DRSUM due to GPT-3 being a generative language model. The summaries written by our human doctors have writing structure similar to that of a hybrid summarization model such as DRSUM that is more extractive in nature. This can explain why DRSUM did not show performance gain when using generated data from GPT-3-ENS. The key, however, is that it still did perform on par.

In the same Table 7, we also present the results with increased amounts of data (12800 and 25600) from GPT-3-ENS. There is little or no further improvement in the automated metrics of concept and negation F1. However, ROUGE-L F1 improves reflecting the improvements in coherency of the summaries. We leave this area as future work to explore.

Effect of Combining Human Labeled Data with Data Labeled by GPT-3-ENS

Since GPT-3 relies on limited local priming context (N=21) it may not be agile in providing robust summaries for a multitude of variations in snippets, focusing on the exploitation part of the exploration-exploitation trade-off. We hypothesize that best summaries then will be synthesized by a model trained on a dataset with human and GPT-3-ENS labeled examples. To evaluate this, we introduced a mixing parameter α, the ratio of GPT-3-ENS labeled examples to human labeled examples. For instance, with 6400 human labeled examples, α=0.5 implies the dataset contains 6400 human labeled examples along with 0.5*6400=3200 GPT-3-ENS generated examples. We experiment with α=0.5, 1, 2, 3.

TABLE 8
Input conversation snippets along with summaries
generated by models trained on different data

| | Model trained on | Model trained on | Model trained on |
|---|---|---|---|
| Snippet | H₆₄₀₀ | GCF₆₄₀₀^{K=10} | H₆₄₀₀ + GCF₃₂₀₀^{K=10} |
| DR: Have you | Has not been | Hasn't tested for | Has not been |
| ever been tested | tested for any | any underlying | tested for any |
| for any underlying | underlying health | health conditions | underlying health |
| health conditions | conditions. | such as diabetes, | conditions. Has |
| such as diabetes, | | hypothyroidism or | been told has |
| hypothyroidism or | | polycystic ovarian | prediabetes. |
| polycystic ovarian | | syndrome | |
| syndrome? | | | |
| PT: No | | | |
| PT: I have been | | | |
| told I have | | | |
| prediabetes | | | |
| DR: Do you have | Has pus appearing | Pus appearing | Pus discharge from |
| pus appearing | from the site. | from the site | the site. If bubbles |
| discharge from the | | | pop it leaks out a |
| site? | | | substance. |
| PT: Yes. If the | | | |
| bubbles pop it | | | |
| leaks out a watery | | | |
| substance | | | |

From Table 9, we observe that for both PEGASUS and DRSUM, mixture of human labeled and GPT-3-ENS data consistently improves almost all automated metrics for all α values¹ The lift in metrics is lower for DRSUM, again illustrating the idea we highlighted of GPT-3-ENS data being more amenable to abstractive models such as PEGASUS than for hybrid or extractive-biased models such as DRSUM. Table 8 provides qualitative comparison between summaries generated by each of these models.

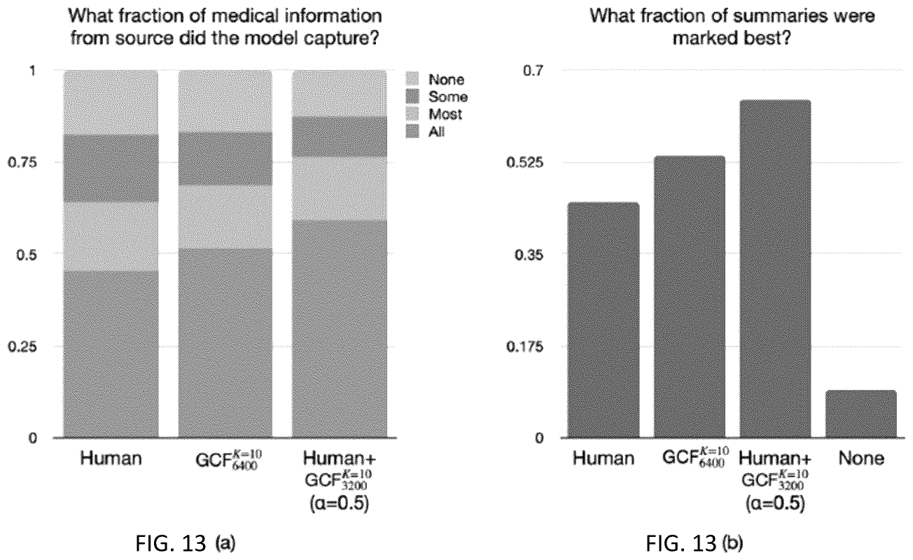

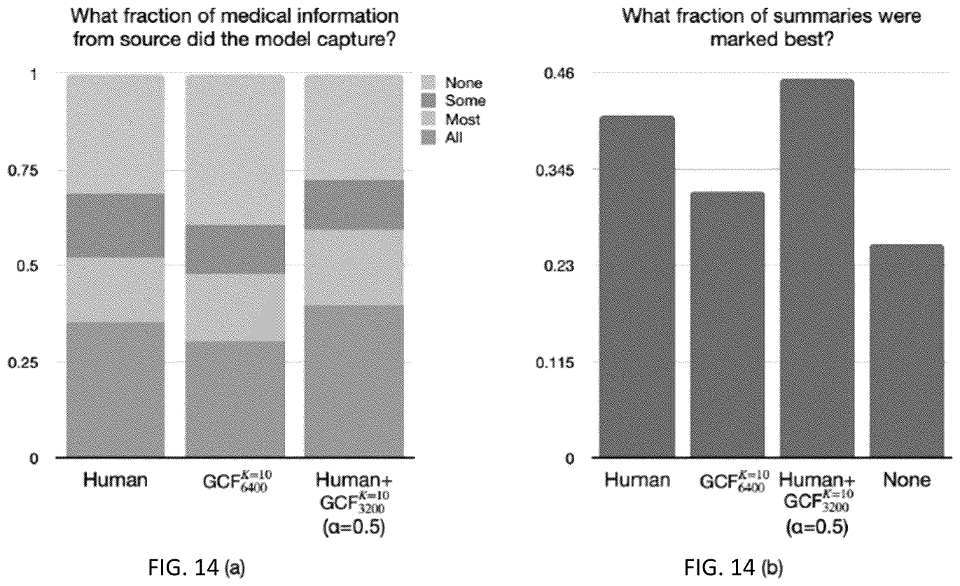

For simplicity, we chose the smallest GPT-3-ENS mix i.e. α=0.5 for human evaluation where we ask doctors to evaluate summaries from model trained on human, GPT-3-ENS and human+GPT-3-ENS data. FIG. 13 and FIG. 14 show that doctors prefer summaries from the model trained on the mixture data over those produced by models trained on human or GPT-3-ENS data alone, in terms of amount of medical information captured as well as the overall quality of the summary. Furthermore, FIG. 13(b) also shows that for PEGASUS, doctors prefer the summaries from a model trained on GCF₆₄₀₀^{K=10} (which needed only 210 human labeled examples) over those produced by a model trained on 6400 human labeled examples.

TABLE 9
Combining human labeled datasets with datasets
generated using our proposed approach

| | | Metrics | | |
|---|---|---|---|---|
| | Train Data | Negation | Concept | ROUGE-L |
| Models | Source | F1 | F1 | F1 |
| PEGASUS | H₆₄₀₀ | 21.09 | 35.96 | 55.59 |
| α = 0.5 | H₆₄₀₀ + GCF₃₂₀₀^{K=10} | 30.14 | 43.49 | 62.45 |
| α = 1 | H₆₄₀₀ + GCF₆₄₀₀^{K=10} | 30.70 | 43.73 | 60.63 |
| α = 2 | H₆₄₀₀ + GCF₁₂₈₀₀^{K=10} | 29.43 | 41.02 | 59.85 |
| α = 3 | H₆₄₀₀ + GCF₂₅₆₀₀^{K=10} | 31.93 | 44.68 | 61.05 |
| DRSUM | H₆₄₀₀ | 26.75 | 39.95 | 52.70 |
| α = 0.5 | H₆₄₀₀ + GCF₃₂₀₀^{K=10} | 27.51 | 40.46 | 53.39 |
| α = 1 | H₆₄₀₀ + GCF₆₄₀₀^{K=10} | 27.18 | 40.36 | 51.00 |
| α = 2 | H₆₄₀₀ + GCF₁₂₈₀₀^{K=10} | 27.19 | 40.68 | 53.07 |
| α = 3 | H₆₄₀₀ + GCF₂₅₆₀₀^{K=10} | 26.33 | 39.89 | 52.29 |

Accordingly, example embodiments provide a medically-aware GPT-3 data labeler, GPT-3-ENS, for the task of medical conversation summarization. A medically aware ensembling criterion that ensembles multiple summaries for an input from a powerful low-shot learner such as GPT-3. We showed that this approach can generate quality training data for medical dialogue summarization models while ensuring medical correctness. We show that using a very small number of human labeled examples, 210, we are able to produce more medically correct and better quality summaries than using roughly thirty times as many human labeled examples for two different summarization models. In this work we used a ensembling technique that dialogue summaries should retain all the medical information discussed in the dialogue.

TABLE 10
Prompt for GPT-3 given two examples

| Snippet | Summary | Prompt |
|---|---|---|
| PT: Today spit out a bit of | Has been on these | Today spit out a bit of |
| mucus and noticed a bit of | medications | mucus and noticed a |
| blood. | for about 2 years. | bit of blood. [STOP] |
| DR: Okay, how long have | | Okay, how long have you |
| you been on these | | been on these |
| medications? | | medications?[SEP] About |
| PT: About 2 years | | 2 years |
| | | [SUMMARIZED] Has |
| | | been on these medications |
| | | for about 2 years. [STOP] |
| DR: Is the bleeding | The bleeding is from the | Is the bleeding from the |
| from the anal opening and | anal opening. | anal opening and not the |
| not the vagina? Has | | vagina? Has something |
| something | | similar happened before? |
| similar happened before? | | [SEP]yes from the anal |
| PT: yes from the anal | | opening[SUMMARIZED] |
| opening | | The bleeding is from the |
| | | anal opening. [STOP] |

GPT-3 Prompt

We utilize a prompt to have GPT-3 generate summaries. Each example (snippet_text, summary_text) is concatenated to the empty string with the following transformation:

“(snippet_text)[SUMMARIZED](summary_text)[STOP]” to form the prompt We separate the conversational turns in snippet_text with the “[SEP]” token. Table 10 shows a prompt that would be generated and used to prime GPT-3 given two examples. As mentioned in § 8 in our experiments we use 21 examples to generate a prompt

### LLM Summaries Component 140

Alternatively, medical summaries can be generated without the training mentioned in the above example embodiments. An example embodiment of the component 140 performs medical conversation summarization by discretizing the task into several smaller dialogue-understanding tasks that are sequentially built upon. First, the component 140 identifies medical entities and their affirmations within the conversation to serve as building blocks. The component 140 then dynamically constructs few-shot prompts for tasks by conditioning on relevant patient information and use a machine learning model (e.g. GPT3, GPT4, etc.) as the backbone.

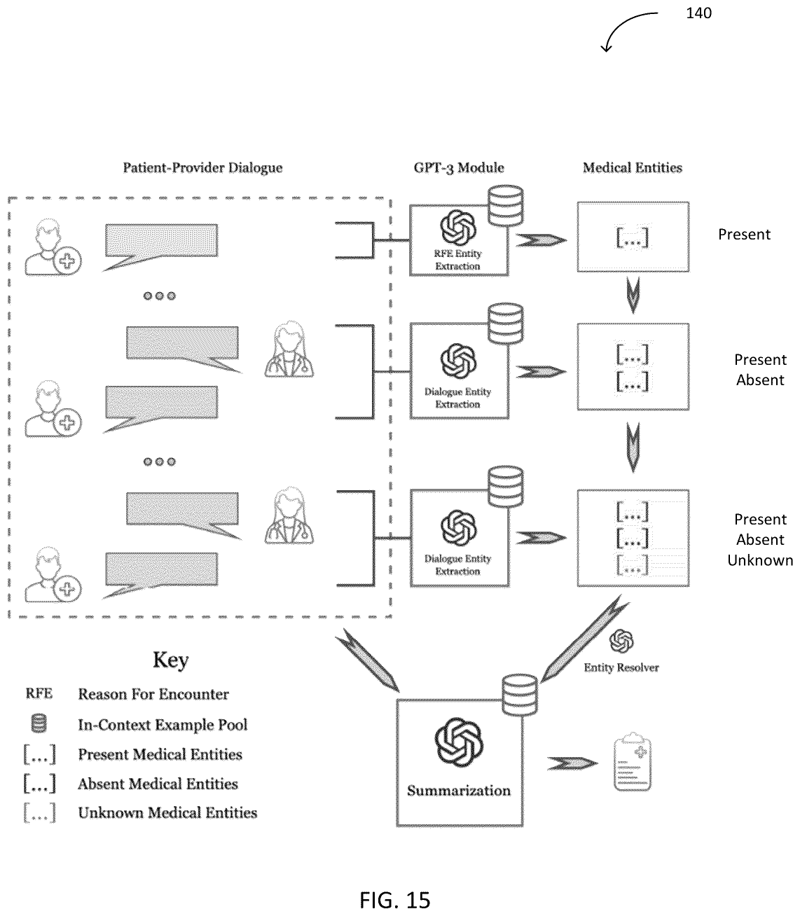

FIG. 15 illustrates an example embodiment of the LLM Summaries 140 (also referred to as MEDSUM-ENT), which grounds the task by first extracting medical entities and their affirmations. These extractions are included as additional input that informs the final summarization step through prompt chaining. MEDSUM-ENT also exploits few-shot prompting for medical concept extraction and summarization through in-context example selection.

In both qualitative physician analysis of medical dialogue summaries and quantitative metrics, MEDSUM-ENT generates clinically accurate summaries and produces summaries that are preferable to a zero-shot, single prompt baseline.
Automated metrics: Quantitative metrics are hard to design for generative tasks. We extend proxy metrics by leveraging GPT-3 to compare the coverage of the presence of medical entities in the generated texts. Beyond only identifying exact matches, our approach better accounts for paraphrasing those medical events within the larger text.

Medical Entity Extraction To highlight clinical concepts, we extract medical entities (symptoms, diseases etc.) and their affirmation status of either present, absent, or unknown. These entities and their status will be used as additional inputs to the final summarization step.

We first perform entity extraction on the patient's first message of the encounter, which is often lengthy and information dense. We call this message the reason for encounter (RFE). Conversational turns between the medical provider and the patient follow the RFE. We also extract medical entities from the conversation, one provider and one patient turn at a time. To accommodate these two types of texts, we use two different prompts, included in Appendix Prompt 1 (for RFE entity extraction) and Appendix Prompt 2 (for dialogue entity extraction). Both prompts are populated with in-context examples (see In-Context Example Selection) along with the patient's age and sex. The final list of entities in the dialogue is obtained by collating all entities extracted across the RFE and all dialogue turns.

Additionally, we also use an entity resolver to resolve entities in the unknown entities list whose status may have changed over the course of the dialogue (see Appendix Prompt 3). For instance, a dialogue turn pair may not have enough information to definitively assign a present or absent status and is thus assigned an entity as “unknown”. A later dialogue turn may contain information that changes that assignment By introducing this refinement step, we reduce mistakes in the “Pertinent Unknowns” section of the summary (see Table 11).

Summarization Given a list of medical entities, we summarize the medical dialogue using the dialogue and the entities as input Our summaries are structured into six sections: Demographics and Social Determinants of Health, Medical Intent, Pertinent Positives, Pertinent Negatives, Pertinent Unknowns, and Medical History (see Appendix Prompt 4 for details).

In-Context Example Selection For the entity extraction and summarization modules, we compare semantic-similarity and random in-context example selection. Semantic-similarity-based selection selects labeled examples from a pool using the patient's age, sex, and the query point. Random selection randomly selects in-context examples from these pools to populate our prompts.

Experiments

Dataset: We use a dataset of 100 clinical encounters of dialogue-summary pairs that occurred between a licensed physician and a patient on a telehealth platform. Encounters in this dataset cover a wide variety of common presentations in telehealth, including urinary tract infections, back/abdominal pains, toothaches, and others. All data was de-identified and scrubbed for protected health information prior to experimentation. Conversations contain 46 dialogue turns on average (e.g., min of 8 turns, max of 92 turns) and an average of 2342 unigram tokens per encounter. Ground truth summaries were created by using text-davinci-002 on encounter data to generate an initial summary, which physicians then edited for correctness.

Baselines/Ablations: We compare MEDSUM-ENT to a “naive” zero-shot, single-prompt baseline (e.g., without chaining) that prompts GPT-3 to summarize the conversation (see Appendix Prompt 5). For MEDSUM-ENT, we evaluate extraction k-shot configurations (1,3,5-shot) and in-context example selection methods (semantic-similarity based, random) for entity extraction. We use RFE and dialogue entity extraction prompts in at least a 1-shot configuration for MEDSUM-ENT to ensure valid output and formatting. Our summarization prompt for baselines and MEDSUM-ENT cannot go beyond 1-shot due to token limit constraints. All experiments are run once and leverage GPT-3 (davinci-003) for generation (see Appendix A.2 for temperature, max_tokens, and top_p settings for each prompt).

Evaluation Metrics

Expert Evaluation We also asked four doctors, who serve patients on a telehealth platform, to judge between the MEDSUM-ENT and baseline-generated summaries on three points on a random set of 50 encounters. For a given encounter, we asked 1) for preference between baseline and MEDSUM-ENT summaries, 2) what amount of clinical information was captured in MEDSUM-ENT's summaries, and 3) about the presence of clinically harmful information in MEDSUM-ENT summaries (see Appendix A.3 for exact instructions and other details).

GPT-Driven Automated Summarization Metrics: Acknowledging the challenges in automatic evaluations of summarization, we focus on quantitatively evaluating the correctness/faithfulness of capturing medical concepts and their affirmation status.

We extend the approach to metrics to have two components, both powered by GPT-3: a medical concept extractor (Appendix Prompt 6) and a verifier (Appendix Prompt 7). The verifier checks if the concepts extracted from one piece of text are present in another and permits the same medical concept extracted or written in different ways to count towards a true positive. For example, for the “Pertinent Positives” section, the predicted value may be “Patient has back pain and COVID-19” and the resulting concepts [“back pain”, “COVID-19” ] and the ground-truth “Patient has COVID and some pain in the backside” with concepts [“COVID”, “pain in the back” ]. Prior metrics that rely on verbatim matches would fail to recognize the predicted text as correct. We define the following metrics:
GPT-Recall: We extract medical entities from both the predicted text and ground-truth text of the same summary section. We use the verifier to infer if the entities extracted from the ground-truth section are also present in the predicted text. This produces tp_{gt} and fₙ, which is used to calculate

$$GPT - Recall = \frac{{tp}_{ℊ t}}{{tp}_{ℊ t} + {f}_{n}} .$$

GPT-Precision: We verify concepts extracted from the predicted section are also present in the ground-truth text, either as exact matches or rephrasings. This produces tp_{pred} and fₚ, which is used to calculate

$$GPT - Precision = \frac{{tp}_{pred}}{{tp}_{pred} + {f}_{p}} .$$

GPT-F1 is the harmonic mean of GPT-Precision and GPT-Recall. Note our approach maintains the integrity of recall and precision (neither score can take on a value >1). We evaluate MEDSUM-ENT via the GPT-Precision and GPT-Recall metrics described in section 3.1 on all 100 clinical encounters.

Results

Table 11 shows quantitative metrics on summaries produced by the baselines and MEDSUM-ENT. Both generated summaries are compared to the ground truth summaries. We see that while GPT-F1 performance for “Pertinent Positives” and “Pertinent Negatives” is consistent across methods, MEDSUM-ENT's ability to capture the “Pertinent Unknowns” and “Medical History” pushes its average consistently above that of the naive zero-shot, non-chained baseline. These sections are crucial to include correctly as they often influence clinical decision-making. Also, the Unknown Entity Resolver improves performance specifically in the “Pertinent Unknowns” section (ablated in rows 7 vs. 8 with 46.4 vs. 55.8 for with and without the resolver). The “Demographics and Social Determinants of Health” and “Medical Intent” sections have nearly identical, accurate output across all experiments, so we do not calculate metrics for them. See Appendix A.4 for example generated summaries.

TABLE 11

| | Extraction | Summarization | Example | Entity | Pertinent | Pertinent | Pertinent | Medical | |
|---|---|---|---|---|---|---|---|---|---|
| Method | K-shot | K-shot | Selection | Resolver | Positives | Negatives | Unknowns | History | Average |
| Naive | — | 0-shot | — | — | 72.9 | 71.7 | 45.4 | 43.9 | 58.5 |
| | — | 1-shot | semantic | — | 71.0 | 69.5 | 42.1 | 48.3 | 57.7 |
| | — | 1-shot | random | — | 69.4 | 69.1 | 47.5 | 44.7 | 57.7 |
| MEDSUM- | 1-shot | 1-shot | semantic | √ | 72.4 | 70.1 | 50.0 | 46.2 | 59.7 |
| ENT | | | | | | | | | |
| | 1-shot | 1-shot | random | √ | 71.4 | 71.1 | 54.0 | 48.3 | 61.2 |
| | 3-shot | 1-shot | semantic | √ | 71.9 | 69.0 | 42.5 | 47.0 | 57.6 |
| | 3-shot | 1-shot | random | — | 72.1 | 69.4 | 46.4 | 45.8 | 58.4 |
| | 3-shot | 1-shot | random | √ | 72.2 | 70.9 | 55.8 | 50.4 | 62.3 |
| | 5-shot | 1-shot | semantic | √ | 71.8 | 70.2 | 46.6 | 46.3 | 58.7 |
| | 5-shot | 1-shot | random | √ | 71.9 | 68.3 | 51.9 | 48.2 | 60.0 |

Results of GPT-driven metrics. Performance across “Pertinent Positives”, “Pertinent Negatives” sections are fairly consistent across methods. MEDSUM-ENT demonstrates consistently improved performance in the “Pertinent Unknowns” and “Medical History” sections. Surprisingly, we also find consistently higher performance across experiments using random in-context example selection over semantic-similarity-based selection.

We find two surprising results. First, there is no correlation between a larger k-shot and increased performance. This may demonstrate diminishing returns of GPT-3 to perform medical concept extraction. Furthermore, the use of semantic similarity to select in-context examples performs worse than randomly selecting examples. This may imply diversity of in-context samples is more important than similarity.

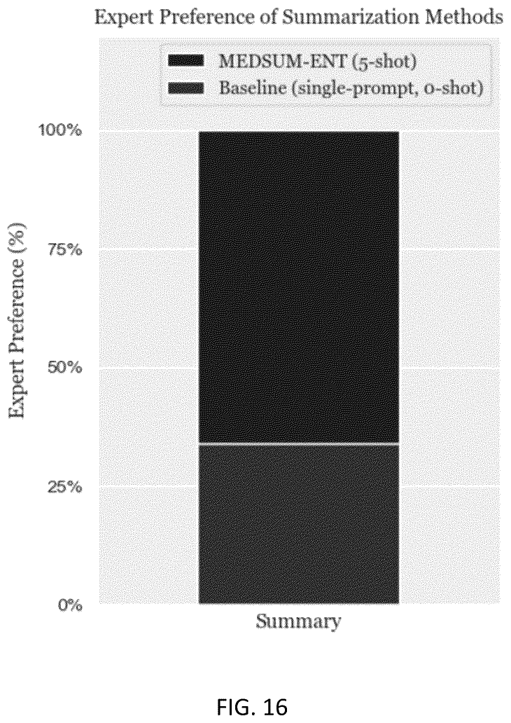

In our expert human evaluations, FIG. 16 demonstrates MEDSUM-ENT (5-shot, semantic) summaries are preferred over the baseline summaries 66% to 34%. Our expert evaluators also rate MEDSUM-ENT capturing all relevant medical information in 40% of evaluated summaries, most information in 48%, some information in 12%, and zero information in 0%. This provides further qualitative evidence for MEDSUM-ENT's ability to effectively summarize. However, our expert evaluators also rate 28% of the summaries evaluated as containing incorrect information that could harm the patient if acted on by medical providers. Often these are due to misattributed symptoms and conditions (e.g., symptoms marked as absent but were present, missed medication allergies). This is consistent with the GPT-F1 measures for pertinent positives and negatives in Table 11 and highlights the challenge involved in deploying a system such as MEDSUM-ENT. Further work is needed to trust such systems in the wild.

### Appendix

### A.1 Dynamic Example Selection

We create labeled in-context example pools for RFE entity extraction and dialogue entity extraction using physician labels for what medical concepts would have been extracted and created a summarization pool using physician-written dialogue summaries. The dialogue summaries for this pool were created by physicians editing the outputs of summaries created by text-davinci-002. Semantic-similarity based example selection is implemented using nearest-neighbor search with the LangChain and FAISS libraries.

### A.2 Experiment Details

TABLE 12
Experimental settings for all prompts used in this work, no hyper-
parameter search was run to obtain these values. We use lower
temperature values for model calls where we expect lower variability
in its inputs (summarization takes in dialogues and list of
medical entities of varying lengths and sizes respectively,
thus has a higher temperature). Running the metric concept extraction
and verification prompts at a temperature of 0 ensures maximal
reproducibility of metric computation. Each experiment (line
in Table 11 took approximately 3 hours to run, with exponential
back-off used during GPT-3 queries.)

| Prompt | temperature | max_tokens | top_p |
|---|---|---|---|
| RFE Medical Entity Extr. | 0.1 | 200 | 1.0 |
| Dialogue Medical Entity Extr. | 0.1 | 200 | 1.0 |
| Unknown Entity Resolver | 0.1 | 200 | 1.0 |
| Summarization | 0.7 | 512 | 1.0 |
| Metric: Medical Entity Extr. | 0.0 | 200 | 1.0 |
| Metric: Medical Entity Verif. | 0.0 | 200 | 1.0 |

### A.3 Expert Evaluation

To qualitatively evaluate our summaries, we conducted physician evaluations focused on three questions:

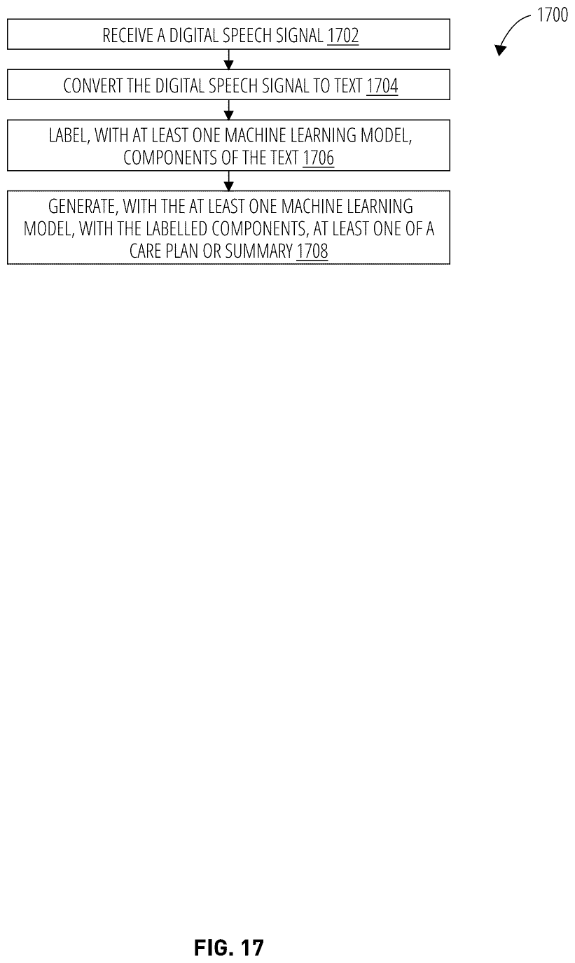

FIG. 17 illustrates an example routine 1700 for speech processing with an artificial intelligence. In block 1702, at least one processor optionally receives a digital speech signal. In block 1704, the at least one processor optionally converts the digital speech signal to text. In block 1706, the at least one processor labels, with at least one machine learning model, components of the text. In block 1708, the at least one processor generates, with the at least one machine learning model, with the labelled components, at least one of a care plan or summary.

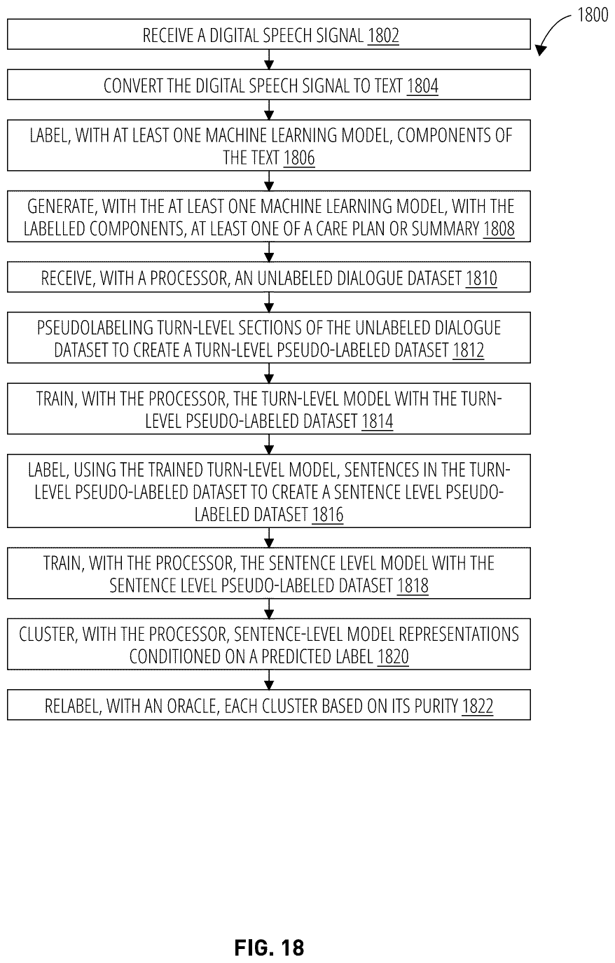

FIG. 18 illustrates an example routine 1800 for speech processing with an artificial intelligence. In block 1802, the at least one processor receives a digital speech signal. In block 1804, the at least one processor converts the digital speech signal to text. In block 1806, the at least one processor labels, with at least one machine learning model, components of the text. In block 1808, the at least one processor generates, with the at least one machine learning model, with the labelled components, at least one of a care plan or summary. In block 1810, the at least one processor receives an unlabeled dialogue dataset. In block 1812, routine 1800 pseudolabeling turn-level sections of the unlabeled dialogue dataset to create a turn-level pseudo-labeled dataset. In block 1814, the at least one processor trains the turn-level model with the turn-level pseudo-labeled dataset. In block 1816, the at least one processor labels, using the trained turn-level model, sentences in the turn-level pseudo-labeled dataset to create a sentence level pseudo-labeled dataset. In block 1818, the at least one processor trains the sentence level model with the sentence level pseudo-labeled dataset. In block 1820, the at least one processor clusters sentence-level model representations conditioned on a predicted label. In block 1822, routine 1800 relabels, with an oracle, each cluster based on its purity.

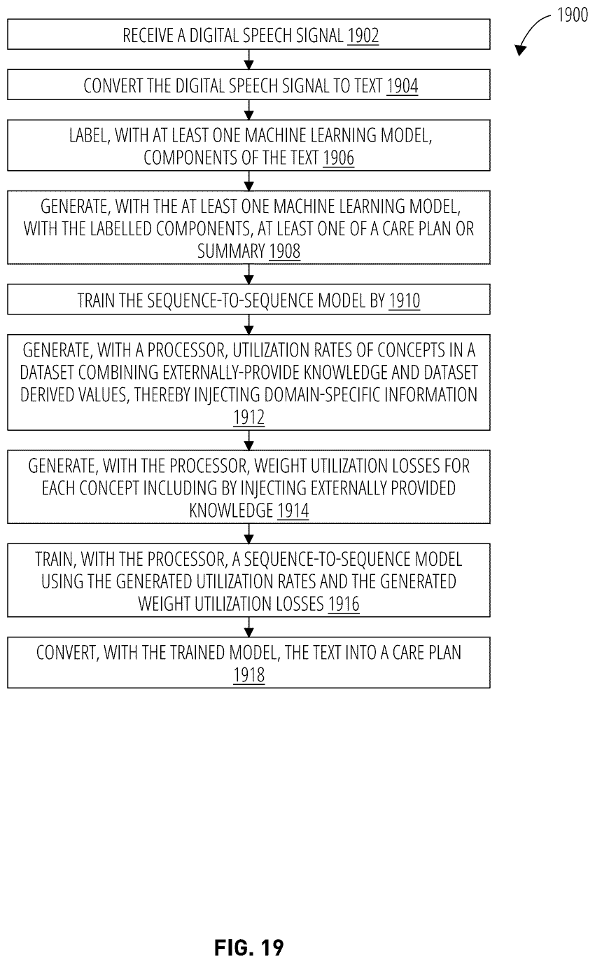

FIG. 19 illustrates an example routine 1900 for speech processing with an artificial intelligence. In block 1902, the at least one processor receives a digital speech signal. In block 1904, the at least one processor converts the digital speech signal to text. In block 1906, the at least one processor labels, with at least one machine learning model, components of the text. In block 1908, the at least one processor generates, with the at least one machine learning model, with the labelled components, at least one of a care plan or summary. In block 1910, the at least one processor trains the sequence-to-sequence model by. In block 1912, the at least one processor generates utilization rates of concepts in a dataset combining externally-provide knowledge and dataset derived values, thereby injecting domain-specific information. In block 1914, the at least one processor generates weight utilization losses for each concept including by injecting externally provided knowledge. In block 1916, the at least one processor trains a sequence-to-sequence model using the generated utilization rates and the generated weight utilization losses. In block 1918, the at least one processor converts, with the trained model, the text into a care plan.

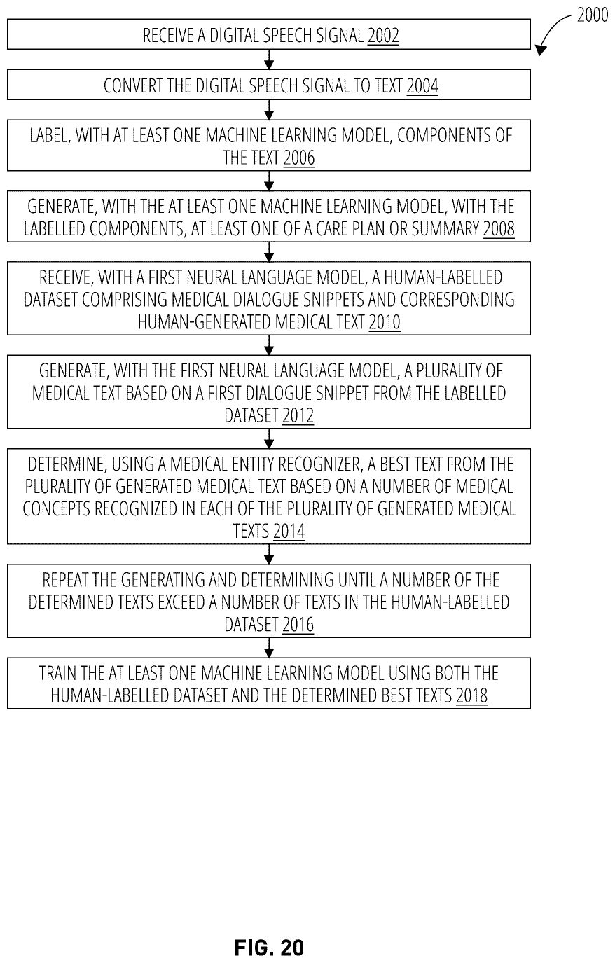

FIG. 20 illustrates an example routine 2000 for speech processing with an artificial intelligence. In block 2002, In block 2002, the at least one processor receives a digital speech signal. In block 2004, the at least one processor converts the digital speech signal to text. In block 2006, the at least one processor labels, with at least one machine learning model, components of the text. In block 2008, the at least one processor generates, with the at least one machine learning model, with the labelled components, at least one of a care plan or summary. In block 2010, the at least one processor receives, with a first neural language model, a human-labelled dataset comprising medical dialogue snippets and corresponding human-generated medical text. In block 2012, the at least one processor generates, with the first neural language model, a plurality of medical text based on a first dialogue snippet from the labelled dataset. In block 2014, the at least one processor determines, using a medical entity recognizer, a best text from the plurality of generated medical text based on a number of medical concepts recognized in each of the plurality of generated medical texts. In block 2016, the at least one processor repeats the generating and determining until a number of the determined texts exceed a number of texts in the human-labelled dataset. In block 2018, the at least one processor trains the at least one machine learning model using both the human-labelled dataset and the determined best texts.

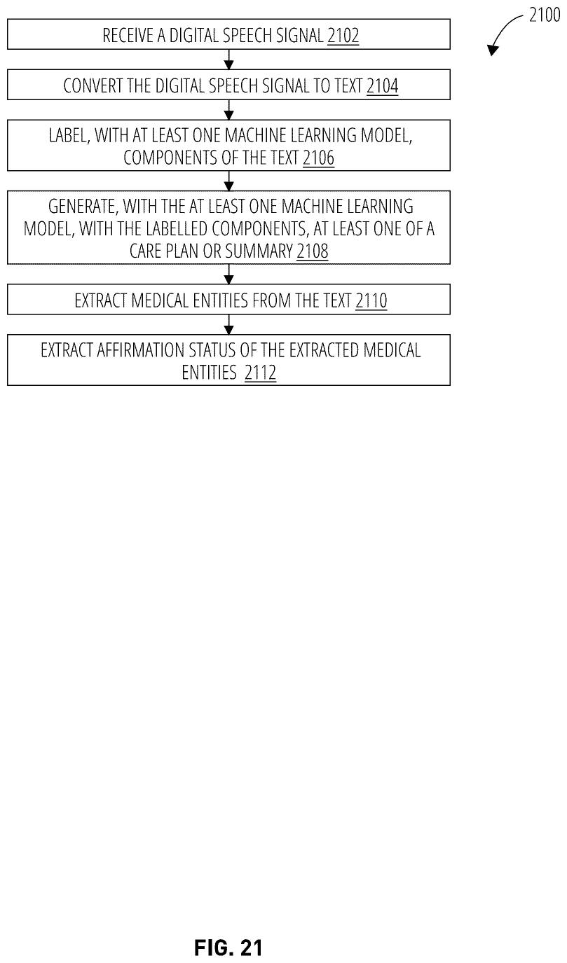

FIG. 21 illustrates an example routine 2100 for speech processing with an artificial intelligence. In block 2102, the at least one processor receives a digital speech signal. In block 2104, the at least one processor converts the digital speech signal to text. In block 2106, the at least one processor labels, with at least one machine learning model, components of the text. In block 2108, the at least one processor generates, with the at least one machine learning model, with the labelled components, at least one of a care plan or summary. In block 2110, the at least one processor extracts medical entities from the text. In block 2112, the at least one processor extracts affirmation status of the extracted medical entities.

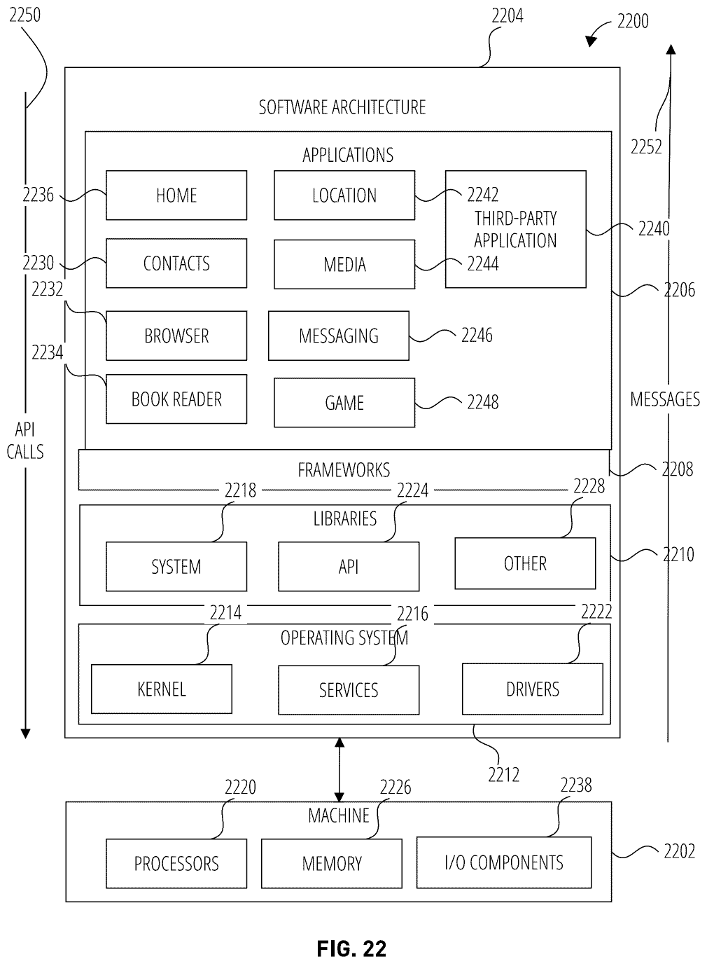

FIG. 22 is a block diagram 2200 illustrating a software architecture 2204, which can be installed on any one or more of the devices described herein. The software architecture 2204 is supported by hardware such as a machine 2202 that includes processors 2220, memory 2226, and I/O components 2238. In this example, the software architecture 2204 can be conceptualized as a stack of layers, where each layer provides a particular functionality. The software architecture 2204 includes layers such as an operating system 2212, libraries 2210, frameworks 2208, and applications 2206. Operationally, the applications 2206 invoke API calls 2250 through the software stack and receive messages 2252 in response to the API calls 2250.

The operating system 2212 manages hardware resources and provides common services. The operating system 2212 includes, for example, a kernel 2214, services 2216, and drivers 2222. The kernel 2214 acts as an abstraction layer between the hardware and the other software layers. For example, the kernel 2214 provides memory management, Processor management (e.g., scheduling), component management, networking, and security settings, among other functionalities. The services 2216 can provide other common services for the other software layers. The drivers 2222 are responsible for controlling or interfacing with the underlying hardware. For instance, the drivers 2222 can include display drivers, camera drivers, BLUETOOTH® or BLUETOOTH® Low Energy drivers, flash memory drivers, serial communication drivers (e.g., Universal Serial Bus (USB) drivers), WI-FI® drivers, audio drivers, and power management drivers.

The libraries 2210 provide a low-level common infrastructure used by the applications 2206. The libraries 2210 can include system libraries 2218 (e.g., C standard library) that provide functions such as memory allocation functions, string manipulation functions, mathematic functions, and the like. In addition, the libraries 2210 can include API libraries 2224 such as media libraries (e.g., libraries to support presentation and manipulation of various media formats such as Moving Picture Experts Group-4 (MPEG4), Advanced Video Coding (H.264 or AVC), Moving Picture Experts Group Layer-3 (MP3), Advanced Audio Coding (AAC), Adaptive Multi-Rate (AMR) audio codec, Joint Photographic Experts Group (JPEG or JPG), or Portable Network Graphics (PNG)), graphics libraries (e.g., an OpenGL framework used to render in two dimensions (2D) and three dimensions (3D) in a graphic content on a display), database libraries (e.g., SQLite to provide various relational database functions), web libraries (e.g., Web Kit to provide web browsing functionality), and the like. The libraries 2210 can also include a wide variety of other libraries 2228 to provide many other APIs to the applications 2206.

The frameworks 2208 provide a high-level common infrastructure used by the applications 2206. For example, the frameworks 2208 provide various graphical user interface (GUI) functions, high-level resource management, and high-level location services. The frameworks 2208 can provide a broad spectrum of other APIs that can be used by the applications 2206, some of which may be specific to a particular operating system or platform.

In some examples, the applications 2206 may include a home application 2236, a contacts application 2230, a browser application 2232, a book reader application 2234, a location application 2242, a media application 2244, a messaging application 2246, a game application 2248, and a broad assortment of other applications such as a third-party application 2240. The applications 2206 are programs that execute functions defined in the programs. Various programming languages can be employed to create one or more of the applications 2206, structured in a variety of manners, such as object-oriented programming languages (e.g., Objective-C, Java, or C++) or procedural programming languages (e.g., C or assembly language).In a specific example, the third-party application 2240 (e.g., an application developed using the ANDROID™ or IOS™ software development kit (SDK) by an entity other than the vendor of the particular platform) may be mobile software running on a mobile operating system such as IOS™, ANDROID™, WINDOWS® Phone, or another mobile operating system. In this example, the third-party application 2240 can invoke the API calls 2250 provided by the operating system 2212 to facilitate functionality described herein.

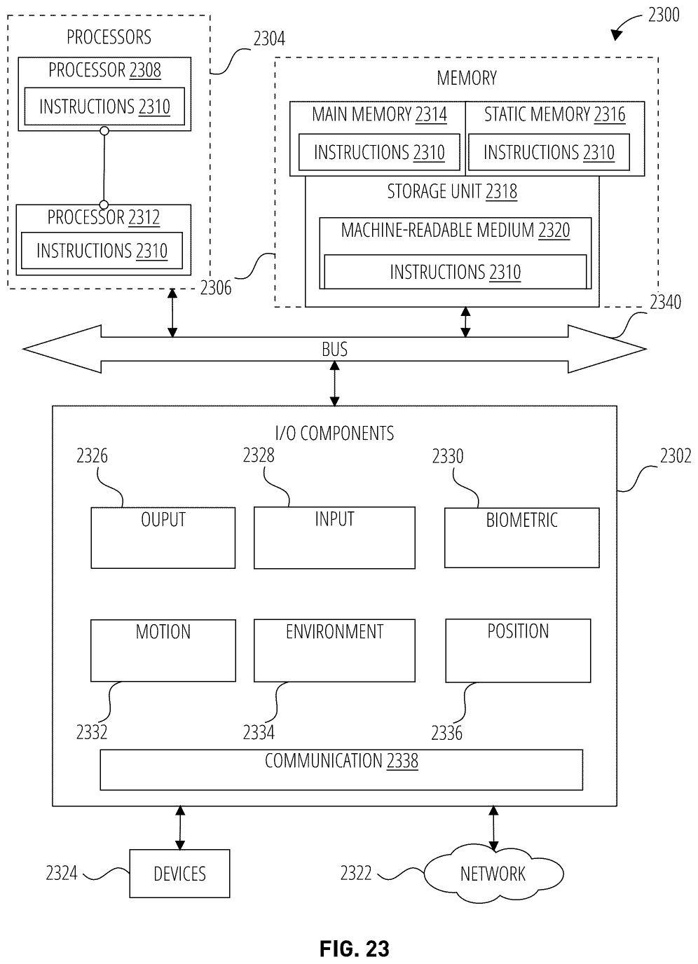

FIG. 23 is a diagrammatic representation of the machine 2300 within which instructions 2310 (e.g., software, a program, an application, an applet, an app, or other executable code) for causing the machine 2300 to perform any one or more of the methodologies discussed herein may be executed. For example, the instructions 2310 may cause the machine 2300 to execute any one or more of the methods described herein. The instructions 2310 transform the general, non-programmed machine 2300 into a particular machine 2300 programmed to carry out the described and illustrated functions in the manner described. The machine 2300 may operate as a standalone device or be coupled (e.g., networked) to other machines. In a networked deployment, the machine 2300 may operate in the capacity of a server machine or a client machine in a server-client network environment, or as a peer machine in a peer-to-peer (or distributed) network environment. The machine 2300 may comprise, but not be limited to, a server computer, a client computer, a personal computer (PC), a tablet computer, a laptop computer, a netbook, a set-top box (STB), an entertainment media system, a cellular telephone, a smartphone, a mobile device, a wearable device (e.g., a smartwatch), a smart home device (e.g., a smart appliance), other smart devices, a web appliance, a network router, a network switch, a network bridge, or any machine capable of executing the instructions 2310, sequentially or otherwise, that specify actions to be taken by the machine 2300. Further, while a single machine 2300 is illustrated, the term “machine” may include a collection of machines that individually or jointly execute the instructions 2310 to perform any one or more of the methodologies discussed herein.

The machine 2300 may include processors 2304, memory 2306, and I/O components 2302, which may be configured to communicate via a bus 2340. In some examples, the processors 2304 (e.g., a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), an Application-Specific Integrated Circuit (ASIC), a Radio-Frequency Integrated Circuit (RFIC), another Processor, or any suitable combination thereof) may include, for example, a Processor 2308 and a Processor 2312 that execute the instructions 2310. The term “Processor” is intended to include multi-core processors that may comprise two or more independent processors (sometimes referred to as “cores”) that may execute instructions contemporaneously. Although FIG. 23 shows multiple processors 2304, the machine 2300 may include a single processor with a single core, a single processor with multiple cores (e.g., a multi-core processor), multiple processors with a single core, multiple processors with multiples cores, or any combination thereof.

The memory 2306 includes a main memory 2314, a static memory 2316, and a storage unit 2318, both accessible to the processors 2304 via the bus 2340. The main memory 2306, the static memory 2316, and storage unit 2318 store the instructions 2310 embodying any one or more of the methodologies or functions described herein. The instructions 2310 may also reside, wholly or partially, within the main memory 2314, within the static memory 2316, within machine-readable medium 2320 within the storage unit 2318, within the processors 2304 (e.g., within the processor's cache memory), or any suitable combination thereof, during execution thereof by the machine 2300.

The I/O components 2302 may include various components to receive input, provide output, produce output, transmit information, exchange information, or capture measurements. The specific I/O components 2302 included in a particular machine depend on the type of machine. For example, portable machines such as mobile phones may include a touch input device or other such input mechanisms, while a headless server machine will likely not include such a touch input device. The I/O components 2302 may include many other components not shown in FIG. 23. In various examples, the I/O components 2302 may include output components 2326 and input components 2328. The output components 2326 may include visual components (e.g., a display such as a plasma display panel (PDP), a light-emitting diode (LED) display, a liquid crystal display (LCD), a projector, or a cathode ray tube (CRT)), acoustic components (e.g., speakers), haptic components (e.g., a vibratory motor, resistance mechanisms), or other signal generators. The input components 2328 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further examples, the I/O components 2302 may include biometric components 2330, motion components 2332, environmental components 2334, or position components 2336, among a wide array of other components. For example, the biometric components 2330 include components to detect expressions (e.g., hand expressions, facial expressions, vocal expressions, body gestures, or eye-tracking), measure biosignals (e.g., blood pressure, heart rate, body temperature, perspiration, or brain waves), or identify a person (e.g., voice identification, retinal identification, facial identification, fingerprint identification, or electroencephalogram-based identification). The motion components 2332 include acceleration sensor components (e.g., accelerometer), gravitation sensor components, rotation sensor components (e.g., gyroscope). The environmental components 2334 include, for example, one or cameras, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 2336 include location sensor components (e.g., a Global Positioning System (GPS) receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 2302 further include communication components 2338 operable to couple the machine 2300 to a network 2322 or devices 2324 via respective coupling or connections. For example, the communication components 2338 may include a network interface Component or another suitable device to interface with the network 2322. In further examples, the communication components 2338 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 2324 may be another machine or any of a wide variety of peripheral devices (e.g., a peripheral device coupled via a USB).

Moreover, the communication components 2338 may detect identifiers or include components operable to detect identifiers. For example, the communication components 2338 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Data glyph, Maxi Code, PDF417, Ultra Code, UCC RSS-2D bar code, and other optical codes), or acoustic detection components (e.g., microphones to identify tagged audio signals). In addition, a variety of information may be derived via the communication components 2338, such as location via Internet Protocol (IP) geolocation, location via Wi-Fi® signal triangulation, or location via detecting an NFC beacon signal that may indicate a particular location.

The various memories (e.g., main memory 2314, static memory 2316, and/or memory of the processors 2304) and/or storage unit 2318 may store one or more sets of instructions and data structures (e.g., software) embodying or used by any one or more of the methodologies or functions described herein. These instructions (e.g., the instructions 2310), when executed by processors 2304, cause various operations to implement the disclosed examples.

The instructions 2310 may be transmitted or received over the network 2322, using a transmission medium, via a network interface device (e.g., a network interface component included in the communication components 2338) and using any one of several well-known transfer protocols (e.g., hypertext transfer protocol (HTTP)). Similarly, the instructions 2310 may be transmitted or received using a transmission medium via a coupling (e.g., a peer-to-peer coupling) to the devices 2324.

### Examples

1. A method of speech signal processing using artificial intelligence, comprising:
optionally receiving, with at least one processor, a digital speech signal; optionally converting, with the at least one processor, the digital speech signal to text;
labelling, with at least one machine learning model, components of the text; generating, with the at least one machine learning model, with the labelled components, at least one of a care plan or summary.

2. The method of example 1, wherein the at least one machine learning model includes a turn-level model and a sentence-level model and further comprising training the turn-level model and the sentence-level model by:
receiving, with the at least one processor, an unlabeled dialogue dataset; pseudolabeling turn-level sections of the unlabeled dialogue dataset to create a turn-level pseudo-labeled dataset;
training, with the at least one processor, the turn-level model with the turn-level pseudo-labeled dataset;
labelling, using the trained turn-level model, sentences in the turn-level pseudo-labeled dataset to create a sentence level pseudo-labeled dataset; training, with the at least one processor, the sentence level model with the sentence level pseudo-labeled dataset;
clustering, with the at least one processor, sentence-level model representations conditioned on a predicted label; and relabeling, with an oracle, each cluster based on its purity.

3. The method of any of the preceding examples, further comprising iteratively refining the sentence level pseudo-labeled dataset with the relabeled clusters and retraining the sentence level model with the refined sentence level pseudo-labeled dataset.

4. The method of any of the preceding examples, wherein the labels include history taking, summarization, education, care plan and other.

5. The method of any of the preceding examples, further comprising removing data labelled as none from the pseudo-labeled dataset.

6. The method of any of the preceding examples, wherein the pseudolabeling uses task-specific heuristics.

7. The method of any of the preceding examples, wherein the task-specific heuristics include embedding turns into fixed-sized representations by mean-pooling a final layer of a sentence encoder.

8. The method of any of the preceding examples, wherein the task-specific heuristics include using a rule-based labeler for identifying summarization turns by string matching.

9. The method of any of the preceding examples, wherein the turn-level model comprises a single feed-forward layer with sigmoidal activation for each label.

10. The method of any of the preceding examples, wherein the at least one machine learning model includes a sequence-to-sequence model and further comprising training the sequence-to-sequence model by:
generating, with the at least one processor, utilization rates of concepts in a dataset combining externally-provide knowledge and dataset derived values, thereby injecting domain-specific information;
generating, with the at least one processor, weight utilization losses for each concept including by injecting externally provided knowledge; and
training, with the at least one processor, a sequence-to-sequence model using the generated utilization rates and the generated weight utilization losses; and converting, with the trained model, the text into a care plan.

11. The method of any of the preceding examples, further comprising recognizing the concepts with a concept recognizer employing a sliding window strategy to find matches of text corresponding to medical concepts and synonyms of the medical concepts.

12. The method of any of the preceding examples, wherein the weight utilization losses are generated for low frequency important concepts.

13. The method of any of the preceding examples, further comprising deriving, with the processor, a dataset of 1-1 mappings of sentences in the text and the care plan and training the model with the mappings.

14. The method of any of the preceding examples, wherein the mappings are based on highest cosine similarity.

15. The method of any of the preceding examples, further comprising generating synthetic medical dialogue training data for the at least one machine learning model, comprising:
receiving, with a first neural language model, a human-labelled dataset comprising medical dialogue snippets and corresponding human-generated medical text;
generating, with the first neural language model, a plurality of medical text based on a first dialogue snippet from the labelled dataset;
determining, using a medical entity recognizer, a best text from the plurality of generated medical text based on a number of medical concepts recognized in each of the plurality of generated medical texts;
repeating the generating and determining until a number of the determined texts exceed a number of texts in the human-labelled dataset; and
training the at least one machine learning model using both the human-labelled dataset and the determined best texts.

16. The method of any of the preceding examples, wherein the first neural language model includes a generative artificial intelligence.

17. The method of any of the preceding examples, further comprising generating the human-labelled dataset by receiving a human-generated summary of a medical dialogue via a graphical user interface and storing the human-generated summary and corresponding dialogue in a non-transitory memory.

18. The method of any of the preceding examples, further comprising generating the human-labelled dataset by using the at least one machine learning model to generate a summary for a dialogue and receiving a human-corrected version of the generated summary.

19. The method of any of the preceding examples, wherein the repeating is continued until the determined summaries exceed the number of summaries if the human-labelled dataset by a factor of thirty.

20. The method of any of the preceding examples, wherein the human-labelled medical text includes medical summaries.

21. The method of any of the preceding examples, wherein the human-labelled medical text includes medical entities.

22. The method of any of the preceding examples, wherein the human-labelled medical text includes triage.

23. The method of any of the preceding examples, wherein the labelling comprises:
extracting medical entities from the text; and
extracting affirmation status of the extracted medical entities.

24. The method of any of the preceding examples, further comprising generating a reason for encounter based on a first message in the text.

25. The method of any of the preceding examples, wherein the extracting medical entities and extracting affirmation status comprises:
submitting at least one prompt including the text to a generative artificial intelligence.

26. The method of any of the preceding examples, further comprising classifying at least one of the extracted medical entities as having an unknown affirmation status and resolving the unknown affirmation status based on a later turn in the text.

27. The method of any of the preceding examples, further comprising generating the summary including demographics, medical intent, pertinent positives, pertinent negatives, pertinent unknowns and medical history.

28. A non-transitory computer-readable medium having stored thereon instructions to cause at least one processor to execute a method of speech signal processing using artificial intelligence, the method comprising: optionally receiving a digital speech signal;
optionally converting the digital speech signal to text;
labelling, with at least one machine learning model, components of the text; and generating, with the at least one machine learning model, with the labelled components, at least one of a care plan or summary.

29. An apparatus for speech signal processing using artificial intelligence, comprising:
optionally a microphone configured to receive speech and convert the received speech to a digital speech signal;
at least one processor; and a non-transitory computer-readable medium having stored thereon instructions to cause the least one processor to execute a method of speech signal processing using artificial intelligence, the method comprising:
optionally receiving the digital speech signal;
optionally converting the digital speech signal to text;
labelling, with at least one machine learning model, components of the text; and generating, with the at least one machine learning model, with the labelled components, at least one of a care plan or summary.

1. A method of speech signal processing using artificial intelligence, comprising:
receiving, with at least one processor, a digital speech signal;
converting, with the at least one processor, the digital speech signal to text;
labelling, with at least one machine learning model, components of the text; and
generating, with the at least one machine learning model, with the labelled components, at least one of a care plan or summary.

2. The method of claim 1, wherein the at least one machine learning model includes a turn-level model and a sentence-level model and further comprising training the turn-level model and the sentence-level model by:
receiving, with the at least one processor, an unlabeled dialogue dataset;
pseudolabeling turn-level sections of the unlabeled dialogue dataset to create a turn-level pseudo-labeled dataset;
training, with the at least one processor, the turn-level model with the turn-level pseudo-labeled dataset;
labelling, using the trained turn-level model, sentences in the turn-level pseudo-labeled dataset to create a sentence level pseudo-labeled dataset;
training, with the at least one processor, the sentence level model with the sentence level pseudo-labeled dataset; and
clustering, with the at least one processor, sentence-level model representations conditioned on a predicted label.

3. The method of claim 2, further comprising iteratively refining the sentence level pseudo-labeled dataset with the clusters and retraining the sentence level model with the refined sentence level pseudo-labeled dataset.

4. The method of claim 2, wherein the labels include history taking, summarization, education, care plan and other.

5. The method of claim 4, further comprising removing data labelled as none from the pseudo-labeled dataset.

6. The method of claim 2, wherein the pseudolabeling uses task-specific heuristics.

7. The method of claim 6, wherein the task-specific heuristics include embedding turns into fixed-sized representations by mean-pooling a final layer of a sentence encoder.

8. The method of claim 6, wherein the task-specific heuristics include using a rule-based labeler for identifying summarization turns by string matching.

9. (canceled)

10. The method of claim 1, wherein the at least one machine learning model includes a sequence-to-sequence model and further comprising
training the sequence-to-sequence model by:
generating, with the at least one processor, utilization rates of concepts in a dataset combining externally-provide knowledge and dataset derived values, thereby injecting domain-specific information;
generating, with the at least one processor, weight utilization losses for each concept including by injecting externally provided knowledge; and
training, with the at least one processor, a sequence-to-sequence model using the generated utilization rates and the generated weight utilization losses; and
converting, with the trained model, the text into a care plan.

11. The method of claim 10, further comprising recognizing the concepts with a concept recognizer employing a sliding window strategy to find matches of text corresponding to medical concepts and synonyms of the medical concepts.

12. The method of claim 10, wherein the weight utilization losses are generated for low frequency important concepts.

13. The method of claim 10, further comprising deriving, with the processor, a dataset of 1-1 mappings of sentences in the text and the care plan and training the model with the mappings.

14. The method of claim 13, wherein the mappings are based on highest cosine similarity.

15. A method of generating synthetic medical dialogue training data for the at least one machine learning model, comprising:
receiving, with a first neural language model, a human-labelled dataset comprising medical dialogue snippets and corresponding human-generated medical text;
generating, with the first neural language model, a plurality of medical text based on a first dialogue snippet from a labelled dataset;
determining, using a medical entity recognizer, a best text from the plurality of generated medical text based on a number of medical concepts recognized in each of the plurality of generated medical texts;
repeating the generating and determining until a number of the determined texts exceed a number of texts in the human-labelled dataset; and
training the at least one machine learning model using both the human-labelled dataset and the determined best texts.

16. The method of claim 15, wherein the first neural language model includes a generative artificial intelligence.

17. The method of claim 15, further comprising generating the human-labelled dataset by receiving a human-generated summary of a medical dialogue via a graphical user interface and storing the human-generated summary and corresponding dialogue in a non-transitory memory.

18. The method of claim 15, further comprising generating the human-labelled dataset by using the at least one machine learning model to generate a summary for a dialogue and receiving a human-corrected version of the generated summary.

19. The method of claim 15, wherein the repeating is continued until the determined texts exceed the number of texts in the human-labelled dataset by a factor of thirty.

20. The method of claim 15, wherein the human-labelled medical text includes medical summaries.

21. The method of claim 15, wherein the human-labelled medical text includes medical entities.

22. The method of claim 15, wherein the human-labelled medical text includes triage.

23. The method of claim 1, wherein the labelling comprises:
extracting medical entities from the text; and
extracting affirmation status of the extracted medical entities.

24. The method of claim 23, further comprising generating a reason for encounter based on a first message in the text.

25. The method of claim 23, wherein the extracting medical entities and extracting affirmation status comprises:
submitting at least one prompt including the text to a generative artificial intelligence.

26. The method of claim 23, further comprising classifying at least one of the extracted medical entities as having an unknown affirmation status and resolving the unknown affirmation status based on a later turn in the text.

27. The method of claim 23, further comprising generating the summary including demographics, medical intent, pertinent positives, pertinent negatives, pertinent unknowns and medical history.

28. A non-transitory computer-readable medium having stored thereon instructions to cause at least one processor to execute a method of speech signal processing using artificial intelligence, the method comprising:
receiving a digital speech signal;
converting the digital speech signal to text;
labelling, with at least one machine learning model, components of the text; and
generating, with the at least one machine learning model, with the labelled components, at least one of a care plan or summary.

29. An apparatus for speech signal processing using artificial intelligence, comprising:
a microphone configured to receive speech and convert the received speech to a digital speech signal;
at least one processor; and
a non-transitory computer-readable medium having stored thereon instructions to cause the least one processor to execute a method of speech signal processing using artificial intelligence, the method comprising:
receiving the digital speech signal;
converting the digital speech signal to text;
labelling, with at least one machine learning model, components of the text; and
generating, with the at least one machine learning model, with the labelled components, at least one of a care plan or summary.

30. The method of claim 1, further comprising generating the summary by extracting entities from the text and confirming, by an oracle, that the summary includes the extracted entities.
